SEQUENCE LISTING (350 sequences)

SEQ ID NO: 1 - DNA, 19 bp, Artificial Sequence
  Description of Artificial Sequence Synthetic primer
ccgaaaaagatgggctttt

SEQ ID NO: 2 - DNA, 23 bp, Artificial Sequence
  Description of Artificial Sequence Synthetic primer
aggttaaaaagtccgaaactatt

SEQ ID NO: 3 - DNA, 23 bp, Artificial Sequence
  Description of Artificial Sequence Synthetic primer
tctcccgatcaaattagaaattg

SEQ ID NO: 4 - DNA, 29 bp, Artificial Sequence
  Description of Artificial Sequence Synthetic primer
aaagagataaaagattttgaaagaataaa

SEQ ID NO: 5 - DNA, 22 bp, Artificial Sequence
  Description of Artificial Sequence Synthetic primer
aaagctaggtttttggagtttt

SEQ ID NO: 6 - DNA, 26 bp, Artificial Sequence
  Description of Artificial Sequence Synthetic primer
acagaaaaagaagaagaattgattaa

SEQ ID NO: 7 - DNA, 21 bp, Artificial Sequence
  Description of Artificial Sequence Synthetic primer
atgatgctgggaatcaggttc

SEQ ID NO: 8 - DNA, 18 bp, Artificial Sequence
  Description of Artificial Sequence Synthetic primer
gggcttggacttgatttg

SEQ ID NO: 9 - DNA, 24 bp, Artificial Sequence
  Description of Artificial Sequence Synthetic primer
gtcttttaatgtgctaatgcaaga

SEQ ID NO: 10 - DNA, 23 bp, Artificial Sequence
  Description of Artificial Sequence Synthetic primer
gcgttcctactaatgtatcaggg

SEQ ID NO: 11 - DNA, 28 bp, Artificial Sequence
  Description of Artificial Sequence Synthetic primer
ggcagagttaaaatatatgaaaatatag

SEQ ID NO: 12 - DNA, 23 bp, Artificial Sequence
  Description of Artificial Sequence Synthetic primer
cacccttcaagaacttttaacag

SEQ ID NO: 13 - DNA, 18 bp, Artificial Sequence
  Description of Artificial Sequence Synthetic primer
ggctcttgaagcttatgg

SEQ ID NO: 14 - DNA, 19 bp, Artificial Sequence
  Description of Artificial Sequence Synthetic primer
agacttggagaaatggagg

SEQ ID NO: 15 - DNA, 19 bp, Artificial Sequence
  Description of Artificial Sequence Synthetic primer
gaggaaaggctcaatttgg

SEQ ID NO: 16 - DNA, 19 bp, Artificial Sequence
  Description of Artificial Sequence Synthetic primer
tcttgtttctcagcaacct

SEQ ID NO: 17 - DNA, 16 bp, Artificial Sequence
  Description of Artificial Sequence Synthetic primer
cgcaagatcaacaggc

SEQ ID NO: 18 - DNA, 23 bp, Artificial Sequence
  Description of Artificial Sequence Synthetic primer
actacaccatcttgttgatgata

SEQ ID NO: 19 - DNA, 21 bp, Artificial Sequence
  Description of Artificial Sequence Synthetic primer
gtaatggttggggtgatttac

SEQ ID NO: 20 - DNA, 16 bp, Artificial Sequence
  Description of Artificial Sequence Synthetic primer
ggagagccgttcgaaa

SEQ ID NO: 21 - DNA, 29 bp, Artificial Sequence
  Description of Artificial Sequence Synthetic primer
ccaacttctaaagaaattttatatgatgg

SEQ ID NO: 22 - DNA, 23 bp, Artificial Sequence
  Description of Artificial Sequence Synthetic primer
aggaaaaattaaaaactgctgga

SEQ ID NO: 23 - DNA, 24 bp, Artificial Sequence
  Description of Artificial Sequence Synthetic primer
tgtttttgaatctgctacaaatga

SEQ ID NO: 24 - DNA, 28 bp, Artificial Sequence
  Description of Artificial Sequence Synthetic primer
ctggtaaatatcttggtgaatcttataa

SEQ ID NO: 25 - DNA, 22 bp, Artificial Sequence
  Description of Artificial Sequence Synthetic primer
ggacagttaatggaatctcaat

SEQ ID NO: 26 - DNA, 24 bp, Artificial Sequence
  Description of Artificial Sequence Synthetic primer
ataccaaatatgagcaactggggc

SEQ ID NO: 27 - DNA, 20 bp, Artificial Sequence
  Description of Artificial Sequence Synthetic primer
aagcccaatcctagagggta

SEQ ID NO: 28 - DNA, 25 bp, Artificial Sequence
  Description of Artificial Sequence Synthetic primer
tagaattcaaactagatgctgtaat

SEQ ID NO: 29 - DNA, 27 bp, Artificial Sequence
  Description of Artificial Sequence Synthetic primer
cggttcaattactacatatttttcata

SEQ ID NO: 30 - DNA, 19 bp, Artificial Sequence
  Description of Artificial Sequence Synthetic primer
gcccggttcaattactaca

SEQ ID NO: 31 - DNA, 24 bp, Artificial Sequence
  Description of Artificial Sequence Synthetic primer
tcttcatttaaaagctgcattttt

SEQ ID NO: 32 - DNA, 23 bp, Artificial Sequence
  Description of Artificial Sequence Synthetic primer
gctctctagcttctatgtactca

SEQ ID NO: 33 - DNA, 26 bp, Artificial Sequence
  Description of Artificial Sequence Synthetic primer
aagcattaaaagacataccatatcgc

SEQ ID NO: 34 - DNA, 24 bp, Artificial Sequence
  Description of Artificial Sequence Synthetic primer
gaagagttttaatagcctcagccc

SEQ ID NO: 35 - DNA, 21 bp, Artificial Sequence
  Description of Artificial Sequence Synthetic primer
gacgaaagctcatcaagatca

SEQ ID NO: 36 - DNA, 30 bp, Artificial Sequence
  Description of Artificial Sequence Synthetic primer
cagttttatcatctttatctatcatttgaa

SEQ ID NO: 37 - DNA, 27 bp, Artificial Sequence
  Description of Artificial Sequence Synthetic primer
aaattctcaataatttcaagacgtctt

SEQ ID NO: 38 - DNA, 21 bp, Artificial Sequence
  Description of Artificial Sequence Synthetic primer
atccactctggcttattgcca

SEQ ID NO: 39 - DNA, 19 bp, Artificial Sequence
  Description of Artificial Sequence Synthetic primer
ggggaataacaggaagaac

SEQ ID NO: 40 - DNA, 15 bp, Artificial Sequence
  Description of Artificial Sequence Synthetic primer
gctgaaccattggcc

SEQ ID NO: 41 - DNA, 15 bp, Artificial Sequence
  Description of Artificial Sequence Synthetic primer
atgttgcaaagcgcc

SEQ ID NO: 42 - DNA, 35 bp, Artificial Sequence
  Description of Artificial Sequence Synthetic primer
cgattatttctatttatgactcttctataaagatc

SEQ ID NO: 43 - DNA, 23 bp, Artificial Sequence
  Description of Artificial Sequence Synthetic primer
gcattaagaagaagcaactttct

SEQ ID NO: 44 - DNA, 27 bp, Artificial Sequence
  Description of Artificial Sequence Synthetic primer
attcttttttcgtttctcacaataatc

SEQ ID NO: 45 - DNA, 24 bp, Artificial Sequence
  Description of Artificial Sequence Synthetic primer
gtcaaaaagagagtctactgattc

SEQ ID NO: 46 - DNA, 24 bp, Artificial Sequence
  Description of Artificial Sequence Synthetic primer
gaacctttgacaacctttctttta

SEQ ID NO: 47 - DNA, 15 bp, Artificial Sequence
  Description of Artificial Sequence Synthetic primer
cgacttgagaggcct

SEQ ID NO: 48 - DNA, 21 bp, Artificial Sequence
  Description of Artificial Sequence Synthetic primer
cctgcttaccttttaatgcat

SEQ ID NO: 49 - DNA, 24 bp, Artificial Sequence
  Description of Artificial Sequence Synthetic primer
ttttaccaagaagattttgcctaa

SEQ ID NO: 50 - DNA, 23 bp, Artificial Sequence
  Description of Artificial Sequence Synthetic primer
caataacagaacgaccagaataa

SEQ ID NO: 51 - DNA, 21 bp, Artificial Sequence
  Description of Artificial Sequence Synthetic primer
gtgttgctatgaatcctgttg

SEQ ID NO: 52 - DNA, 17 bp, Artificial Sequence
  Description of Artificial Sequence Synthetic primer
ggtagaagacccaaggt

SEQ ID NO: 53 - DNA, 20 bp, Artificial Sequence
  Description of Artificial Sequence Synthetic primer
ggaaagctggtaaaagtagg

SEQ ID NO: 54 - DNA, 26 bp, Artificial Sequence
  Description of Artificial Sequence Synthetic primer
tggaaatgaagattatgccaatattt

SEQ ID NO: 55 - DNA, 28 bp, Artificial Sequence
  Description of Artificial Sequence Synthetic primer
tttttaaaaaatgtattgcaacaattgg

SEQ ID NO: 56 - DNA, 20 bp, Artificial Sequence
  Description of Artificial Sequence Synthetic primer
ttatcatctggcgagatgag

SEQ ID NO: 57 - DNA, 19 bp, Artificial Sequence
  Description of Artificial Sequence Synthetic primer
gacgggaattatgtcactg

SEQ ID NO: 58 - DNA, 22 bp, Artificial Sequence
  Description of Artificial Sequence Synthetic primer
ggtggatatgctatgatacttg

SEQ ID NO: 59 - DNA, 19 bp, Artificial Sequence
  Description of Artificial Sequence Synthetic primer
gggtggacagcttataaga

SEQ ID NO: 60 - DNA, 24 bp, Artificial Sequence
  Description of Artificial Sequence Synthetic primer
cgttcacaatattgagcttaatgt

SEQ ID NO: 61 - DNA, 27 bp, Artificial Sequence
  Description of Artificial Sequence Synthetic primer
cttacctcttgaaaatattcctattgg

SEQ ID NO: 62 - DNA, 23 bp, Artificial Sequence
  Description of Artificial Sequence Synthetic primer
ctaatgctccaattaaaattggc

SEQ ID NO: 63 - DNA, 21 bp, Artificial Sequence
  Description of Artificial Sequence Synthetic primer
ggttggagatgttttggaaag

SEQ ID NO: 64 - DNA, 26 bp, Artificial Sequence
  Description of Artificial Sequence Synthetic primer
aaaggtatattatttctcctaaaggc

SEQ ID NO: 65 - DNA, 28 bp, Artificial Sequence
  Description of Artificial Sequence Synthetic primer
gctaatatagctttgcttgtttataaag

SEQ ID NO: 66 - DNA, 26 bp, Artificial Sequence
  Description of Artificial Sequence Synthetic primer
gttgcttctattgaatatgatcctaa

SEQ ID NO: 67 - DNA, 25 bp, Artificial Sequence
  Description of Artificial Sequence Synthetic primer
cgaagagataaatttagcattcctg

SEQ ID NO: 68 - DNA, 26 bp, Artificial Sequence
  Description of Artificial Sequence Synthetic primer
gcataagagaaagtataggttgattg

SEQ ID NO: 69 - DNA, 27 bp, Artificial Sequence
  Description of Artificial Sequence Synthetic primer
ctggtaggattagtattagaagaagag

SEQ ID NO: 70 - DNA, 25 bp, Artificial Sequence
  Description of Artificial Sequence Synthetic primer
aaaaggtaaaaaatttaaatcgggc

SEQ ID NO: 71 - DNA, 24 bp, Artificial Sequence
  Description of Artificial Sequence Synthetic primer
caaaggtaatgatcctttgaaatc

SEQ ID NO: 72 - DNA, 26 bp, Artificial Sequence
  Description of Artificial Sequence Synthetic primer
gctataagacgactttatcttttgat

SEQ ID NO: 73 - DNA, 25 bp, Artificial Sequence
  Description of Artificial Sequence Synthetic primer
agacttataagccaaaaacttcttc

SEQ ID NO: 74 - DNA, 25 bp, Artificial Sequence
  Description of Artificial Sequence Synthetic primer
ggttttggagaaaaataaatatggg

SEQ ID NO: 75 - DNA, 33 bp, Artificial Sequence
  Description of Artificial Sequence Synthetic primer
caaaaaggaagataaaatagatattttttagtg

SEQ ID NO: 76 - DNA, 22 bp, Artificial Sequence
  Description of Artificial Sequence Synthetic primer
tttcttgcgagtcttataacct

SEQ ID NO: 77 - DNA, 38 bp, Artificial Sequence
  Description of Artificial Sequence Synthetic primer
tttatttcttcttttaataataaatttatctgaatatc

SEQ ID NO: 78 - DNA, 28 bp, Artificial Sequence
  Description of Artificial Sequence Synthetic primer
ttttttaatagatcttgccactatactc

SEQ ID NO: 79 - DNA, 31 bp, Artificial Sequence
  Description of Artificial Sequence Synthetic primer
actttttgataaagactcttttctataaaag

SEQ ID NO: 80 - DNA, 20 bp, Artificial Sequence
  Description of Artificial Sequence Synthetic primer
cttctcacttccaaaagacg

SEQ ID NO: 81 - DNA, 25 bp, Artificial Sequence
  Description of Artificial Sequence Synthetic primer
aagatctggagtaggttttaataac

SEQ ID NO: 82 - DNA, 22 bp, Artificial Sequence
  Description of Artificial Sequence Synthetic primer
ggcttaccatttcaggaattat

SEQ ID NO: 83 - DNA, 25 bp, Artificial Sequence
  Description of Artificial Sequence Synthetic primer
aagttttgccattgtaaacagatat

SEQ ID NO: 84 - DNA, 26 bp, Artificial Sequence
  Description of Artificial Sequence Synthetic primer
caagatcctcggtaatataaataggt

SEQ ID NO: 85 - DNA, 17 bp, Artificial Sequence
  Description of Artificial Sequence Synthetic primer
ctcgccaagcttatgtc

SEQ ID NO: 86 - DNA, 22 bp, Artificial Sequence
  Description of Artificial Sequence Synthetic primer
cctctaaaaatccttgtaggtg

SEQ ID NO: 87 - DNA, 22 bp, Artificial Sequence
  Description of Artificial Sequence Synthetic primer
cctctaaaaatccttgtaggtg

SEQ ID NO: 88 - DNA, 23 bp, Artificial Sequence
  Description of Artificial Sequence Synthetic primer
cttccctttttatctgacttagc

SEQ ID NO: 89 - DNA, 29 bp, Artificial Sequence
  Description of Artificial Sequence Synthetic primer
atcttctatttaccaacataactacttac

SEQ ID NO: 90 - DNA, 21 bp, Artificial Sequence
  Description of Artificial Sequence Synthetic primer
agggtaaatttttgccctttg

SEQ ID NO: 91 - DNA, 22 bp, Artificial Sequence
  Description of Artificial Sequence Synthetic primer
ctattggcctaactttttttgg

SEQ ID NO: 92 - DNA, 18 bp, Artificial Sequence
  Description of Artificial Sequence Synthetic primer
agactctccccggatatt

SEQ ID NO: 93 - DNA, 17 bp, Artificial Sequence
  Description of Artificial Sequence Synthetic primer
aaagcactgcaatagcc

SEQ ID NO: 94 - DNA, 24 bp, Artificial Sequence
  Description of Artificial Sequence Synthetic primer
taaaagcttagctcctttattagg

SEQ ID NO: 95 - DNA, 20 bp, Artificial Sequence
  Description of Artificial Sequence Synthetic primer
tgatgctgctgacttaacaa

SEQ ID NO: 96 - DNA, 26 bp, Artificial Sequence
  Description of Artificial Sequence Synthetic primer
ctcggaaagatttttattgtgataca

SEQ ID NO: 97 - DNA, 29 bp, Artificial Sequence
  Description of Artificial Sequence Synthetic primer
catcaaccataactgttttaacaaatatc

SEQ ID NO: 98 - DNA, 21 bp, Artificial Sequence
  Description of Artificial Sequence Synthetic primer
gccaaatctttttacgacgac

SEQ ID NO: 99 - DNA, 19 bp, Artificial Sequence
  Description of Artificial Sequence Synthetic primer
tatcagctctacccctagc

SEQ ID NO: 100 - DNA, 31 bp, Artificial Sequence
  Description of Artificial Sequence Synthetic primer
cttcaacaaaaatatgacaatttctattaac

SEQ ID NO: 101 - DNA, 18 bp, Artificial Sequence
  Description of Artificial Sequence Synthetic primer
cctatccttctgccaacg

SEQ ID NO: 102 - DNA, 28 bp, Artificial Sequence
  Description of Artificial Sequence Synthetic primer
ggaaaaaagattgtatatacttgacatg

SEQ ID NO: 103 - DNA, 26 bp, Artificial Sequence
  Description of Artificial Sequence Synthetic primer
caaagtagaagaagatccaagtattc

SEQ ID NO: 104 - DNA, 24 bp, Artificial Sequence
  Description of Artificial Sequence Synthetic primer
ggcaagaattttgggataataaca

SEQ ID NO: 105 - DNA, 30 bp, Artificial Sequence
  Description of Artificial Sequence Synthetic primer
tgtctaaatacgaattcataaaaattgaaa

SEQ ID NO: 106 - DNA, 30 bp, Artificial Sequence
  Description of Artificial Sequence Synthetic primer
gaataagttgttatgagttaattttcaaga

SEQ ID NO: 107 - DNA, 34 bp, Artificial Sequence
  Description of Artificial Sequence Synthetic primer
taaataaaattaacaaaaatgcaatctaaaagaa

SEQ ID NO: 108 - DNA, 21 bp, Artificial Sequence
  Description of Artificial Sequence Synthetic primer
cacagcatcaaaattgttagc

SEQ ID NO: 109 - DNA, 15 bp, Artificial Sequence
  Description of Artificial Sequence Synthetic primer
ccgtgctggttcaag

SEQ ID NO: 110 - DNA, 14 bp, Artificial Sequence
  Description of Artificial Sequence Synthetic primer
gaggggctagtggg

SEQ ID NO: 111 - DNA, 17 bp, Artificial Sequence
  Description of Artificial Sequence Synthetic primer
ggaagtggtagacacgc

SEQ ID NO: 112 - DNA, 28 bp, Artificial Sequence
  Description of Artificial Sequence Synthetic primer
aaaaaatataatggttaatagtgctgtg

SEQ ID NO: 113 - DNA, 34 bp, Artificial Sequence
  Description of Artificial Sequence Synthetic primer
gtaattaaaaaaaataaaaaagttgacaaaaatt

SEQ ID NO: 114 - DNA, 25 bp, Artificial Sequence
  Description of Artificial Sequence Synthetic primer
cgctgtaatagcaacaacaataata

SEQ ID NO: 115 - DNA, 38 bp, Artificial Sequence
  Description of Artificial Sequence Synthetic primer
aataatattttcaaaaataaaaataattatatttgcaa

SEQ ID NO: 116 - DNA, 22 bp, Artificial Sequence
  Description of Artificial Sequence Synthetic primer
ctctaagcttcaaactaggtca

SEQ ID NO: 117 - DNA, 23 bp, Artificial Sequence
  Description of Artificial Sequence Synthetic primer
aactttgctctcaatagttgttt

SEQ ID NO: 118 - DNA, 32 bp, Artificial Sequence
  Description of Artificial Sequence Synthetic primer
tatgaaatctaatctatttattgtttctgact

SEQ ID NO: 119 - DNA, 22 bp, Artificial Sequence
  Description of Artificial Sequence Synthetic primer
gcaatatttatgtcagcaggaa

SEQ ID NO: 120 - DNA, 21 bp, Artificial Sequence
  Description of Artificial Sequence Synthetic primer
gaaggtttataccctttggag

SEQ ID NO: 121 - DNA, 26 bp, Artificial Sequence
  Description of Artificial Sequence Synthetic primer
tggtcaatatggggtattaactttat

SEQ ID NO: 122 - DNA, 27 bp, Artificial Sequence
  Description of Artificial Sequence Synthetic primer
caataacctgcttgacaaaataaatta

SEQ ID NO: 123 - DNA, 33 bp, Artificial Sequence
  Description of Artificial Sequence Synthetic primer
ggaaattaatgggaaataaattatttaaaaaca

SEQ ID NO: 124 - DNA, 28 bp, Artificial Sequence
  Description of Artificial Sequence Synthetic primer
agacataatatctttttacattgggaaa

SEQ ID NO: 125 - DNA, 21 bp, Artificial Sequence
  Description of Artificial Sequence Synthetic primer
tttaggaattttttggggagc

SEQ ID NO: 126 - DNA, 17 bp, Artificial Sequence
  Description of Artificial Sequence Synthetic primer
tgagggtgagttcctgt

SEQ ID NO: 127 - DNA, 35 bp, Artificial Sequence
  Description of Artificial Sequence Synthetic primer
ttgttttttaaacttattaatattttcttctgtac

SEQ ID NO: 128 - DNA, 15 bp, Artificial Sequence
  Description of Artificial Sequence Synthetic primer
ggcaaaccccaaagc

SEQ ID NO: 129 - DNA, 21 bp, Artificial Sequence
  Description of Artificial Sequence Synthetic primer
gtgttctaatttctcgatccc

SEQ ID NO: 130 - DNA, 25 bp, Artificial Sequence
  Description of Artificial Sequence Synthetic primer
actgtgtccatttatagtaattctc

SEQ ID NO: 131 - DNA, 26 bp, Artificial Sequence
  Description of Artificial Sequence Synthetic primer
ctagcccttttttatataattgcagg

SEQ ID NO: 132 - DNA, 23 bp, Artificial Sequence
  Description of Artificial Sequence Synthetic primer
gtaccatacaggcatttctttta

SEQ ID NO: 133 - DNA, 18 bp, Artificial Sequence
  Description of Artificial Sequence Synthetic primer
ctagtaccgttccaagct

SEQ ID NO: 134 - DNA, 25 bp, Artificial Sequence
  Description of Artificial Sequence Synthetic primer
gtccatctgaagtttgaataatctc

SEQ ID NO: 135 - DNA, 18 bp, Artificial Sequence
  Description of Artificial Sequence Synthetic primer
acgctgtaagatcctctc

SEQ ID NO: 136 - DNA, 29 bp, Artificial Sequence
  Description of Artificial Sequence Synthetic primer
gtaatttttaaaacccactgtcttaaata

SEQ ID NO: 137 - DNA, 19 bp, Artificial Sequence
  Description of Artificial Sequence Synthetic primer
cgtctagcaatctttcagc

SEQ ID NO: 138 - DNA, 22 bp, Artificial Sequence
  Description of Artificial Sequence Synthetic primer
agattcaggccattctaattct

SEQ ID NO: 139 - DNA, 19 bp, Artificial Sequence
  Description of Artificial Sequence Synthetic primer
tccaatttcgctgcatttc

SEQ ID NO: 140 - DNA, 24 bp, Artificial Sequence
  Description of Artificial Sequence Synthetic primer
aattcaatttcaactcctgttgat

SEQ ID NO: 141 - DNA, 18 bp, Artificial Sequence
  Description of Artificial Sequence Synthetic primer
ttttatcgctgtggcctt

SEQ ID NO: 142 - DNA, 19 bp, Artificial Sequence
  Description of Artificial Sequence Synthetic primer
ctttacaacaaggccagac

SEQ ID NO: 143 - DNA, 22 bp, Artificial Sequence
  Description of Artificial Sequence Synthetic primer
cgatcactaaatatgtgatgcc

SEQ ID NO: 144 - DNA, 33 bp, Artificial Sequence
  Description of Artificial Sequence Synthetic primer
gttgttctttgttattttttctattagtttatt

SEQ ID NO: 145 - DNA, 28 bp, Artificial Sequence
  Description of Artificial Sequence Synthetic primer
cttcgtgctttacatattttaaaacatt

SEQ ID NO: 146 - DNA, 22 bp, Artificial Sequence
  Description of Artificial Sequence Synthetic primer
gagaagtcctattaagatcgct

SEQ ID NO: 147 - DNA, 22 bp, Artificial Sequence
  Description of Artificial Sequence Synthetic primer
ctgtgaaaactcccgatttatc

SEQ ID NO: 148 - DNA, 23 bp, Artificial Sequence
  Description of Artificial Sequence Synthetic primer
catttgttattggatgaaatgcg

SEQ ID NO: 149 - DNA, 17 bp, Artificial Sequence
  Description of Artificial Sequence Synthetic primer
gcttccaacccaaattg

SEQ ID NO: 150 - DNA, 17 bp, Artificial Sequence
  Description of Artificial Sequence Synthetic primer
cggttccgtaagttcct

SEQ ID NO: 151 - DNA, 24 bp, Artificial Sequence
  Description of Artificial Sequence Synthetic primer
tctgcttctcaaaatgtaagaaca

SEQ ID NO: 152 - DNA, 24 bp, Artificial Sequence
  Description of Artificial Sequence Synthetic primer
taaccaaatgcacatgttatcaaa

SEQ ID NO: 153 - DNA, 20 bp, Artificial Sequence
  Description of Artificial Sequence Synthetic primer
ttgctgatcaagctcaatat

SEQ ID NO: 154 - DNA, 22 bp, Artificial Sequence
  Description of Artificial Sequence Synthetic primer
gcaacttacagacgaaattaat

SEQ ID NO: 155 - DNA, 24 bp, Artificial Sequence
  Description of Artificial Sequence Synthetic primer
agacagaggttctatacaaattga

SEQ ID NO: 156 - DNA, 21 bp, Artificial Sequence
  Description of Artificial Sequence Synthetic primer
aggtaacggcacatattcaga

SEQ ID NO: 157 - DNA, 23 bp, Artificial Sequence
  Description of Artificial Sequence Synthetic primer
taagaatgaaggaattggcagtt

SEQ ID NO: 158 - DNA, 29 bp, Artificial Sequence
  Description of Artificial Sequence Synthetic primer
aatttaaatgaagtagaaaaagtcttagt

SEQ ID NO: 159 - DNA, 27 bp, Artificial Sequence
  Description of Artificial Sequence Synthetic primer
ggctattaattttattcagacaacaga

SEQ ID NO: 160 - DNA, 22 bp, Artificial Sequence
  Description of Artificial Sequence Synthetic primer
ttgtcacaagcttctagaaata

SEQ ID NO: 161 - DNA, 25 bp, Artificial Sequence
  Description of Artificial Sequence Synthetic primer
tttctggtaagattaatgctcaaat

SEQ ID NO: 162 - DNA, 20 bp, Artificial Sequence
  Description of Artificial Sequence Synthetic primer
gagcttctgatgatgctgct

SEQ ID NO: 163 - DNA, 21 bp, Artificial Sequence
  Description of Artificial Sequence Synthetic primer
gaaaagctttctagtgggtac

SEQ ID NO: 164 - DNA, 24 bp, Artificial Sequence
  Description of Artificial Sequence Synthetic primer
cattaacgctgctaatcttagtaa

SEQ ID NO: 165 - DNA, 23 bp, Artificial Sequence
  Description of Artificial Sequence Synthetic primer
catcagctattaatgcttcaaga

SEQ ID NO: 166 - DNA, 28 bp, Artificial Sequence
  Description of Artificial Sequence Synthetic primer
catggaggaatgatatatgattatcatg

SEQ ID NO: 167 - DNA, 33 bp, Artificial Sequence
  Description of Artificial Sequence Synthetic primer
tttttttttaatttttgtgctattctttttaac

SEQ ID NO: 168 - DNA, 35 bp, Artificial Sequence
  Description of Artificial Sequence Synthetic primer
taataataattatttttaatgctattgctatttgc

SEQ ID NO: 169 - DNA, 28 bp, Artificial Sequence
  Description of Artificial Sequence Synthetic primer
attaaaggcttttgattttaatcaaaga

SEQ ID NO: 170 - DNA, 21 bp, Artificial Sequence
  Description of Artificial Sequence Synthetic primer
ttaagcgcatgaaagatcaag

SEQ ID NO: 171 - DNA, 21 bp, Artificial Sequence
  Description of Artificial Sequence Synthetic primer
gtggaaggtgaacttaatacc

SEQ ID NO: 172 - DNA, 30 bp, Artificial Sequence
  Description of Artificial Sequence Synthetic primer
gattataaaaagaagtacgaagatagagag

SEQ ID NO: 173 - DNA, 33 bp, Artificial Sequence
  Description of Artificial Sequence Synthetic primer
ttatttttttgattaaaaattttcaagtcgtaa

SEQ ID NO: 174 - DNA, 21 bp, Artificial Sequence
  Description of Artificial Sequence Synthetic primer
gcttccggaggagttatttat

SEQ ID NO: 175 - DNA, 18 bp, Artificial Sequence
  Description of Artificial Sequence Synthetic primer
taggagattgtctgtcgc

SEQ ID NO: 176 - DNA, 22 bp, Artificial Sequence
  Description of Artificial Sequence Synthetic primer
gcaacattagctgcataaatat

SEQ ID NO: 177 - DNA, 18 bp, Artificial Sequence
  Description of Artificial Sequence Synthetic primer
tccctcaccagagaaaag

SEQ ID NO: 178 - DNA, 19 bp, Artificial Sequence
  Description of Artificial Sequence Synthetic primer
acaccctcttgaaccggtg

SEQ ID NO: 179 - DNA, 20 bp, Artificial Sequence
  Description of Artificial Sequence Synthetic primer
tgagaaggtgctgtagcagg

SEQ ID NO: 180 - DNA, 27 bp, Artificial Sequence
  Description of Artificial Sequence Synthetic primer
ttgtaacattaacaggagaattaactc

SEQ ID NO: 181 - DNA, 24 bp, Artificial Sequence
  Description of Artificial Sequence Synthetic primer
ttagcaagtgatgtattagcatca

SEQ ID NO: 182 - DNA, 27 bp, Artificial Sequence
  Description of Artificial Sequence Synthetic primer
tgatcacttatcattctaatagcattt

SEQ ID NO: 183 - DNA, 22 bp, Artificial Sequence
  Description of Artificial Sequence Synthetic primer
ctattttggaaagcacctaaat

SEQ ID NO: 184 - DNA, 27 bp, Artificial Sequence
  Description of Artificial Sequence Synthetic primer
gcatactcagtactattctttatagat

SEQ ID NO: 185 - DNA, 24 bp, Artificial Sequence
  Description of Artificial Sequence Synthetic primer
tgagcataagatgcttttagattt

SEQ ID NO: 186 - DNA, 22 bp, Artificial Sequence
  Description of Artificial Sequence Synthetic primer
tctgtcattgtagcatctttta

SEQ ID NO: 187 - DNA, 28 bp, Artificial Sequence
  Description of Artificial Sequence Synthetic primer
ttaaaatactattagttgttgctgctac

SEQ ID NO: 188 - DNA, 18 bp, Artificial Sequence
  Description of Artificial Sequence Synthetic primer
attagcctgcgcaatcat

SEQ ID NO: 189 - DNA, 21 bp, Artificial Sequence
  Description of Artificial Sequence Synthetic primer
gcaatgacaaaacatattggg

SEQ ID NO: 190 - DNA, 34 bp, Artificial Sequence
  Description of Artificial Sequence Synthetic primer
ttaatacaatttataccaattaaactagaatttt

SEQ ID NO: 191 - DNA, 30 bp, Artificial Sequence
  Description of Artificial Sequence Synthetic primer
ataaaaaaacaaaagatcctttaaaggatc

SEQ ID NO: 192 - DNA, 35 bp, Artificial Sequence
  Description of Artificial Sequence Synthetic primer
ataaattatactaaaattattaaatttttgccgat

SEQ ID NO: 193 - DNA, 22 bp, Artificial Sequence
  Description of Artificial Sequence Synthetic primer
gcctgcattatgctttataaca

SEQ ID NO: 194 - DNA, 19 bp, Artificial Sequence
  Description of Artificial Sequence Synthetic primer
cctactcaaagcaaactcc

SEQ ID NO: 195 - DNA, 34 bp, Artificial Sequence
  Description of Artificial Sequence Synthetic primer
cgaaaatactttataacaatctttaattttaaca

SEQ ID NO: 196 - DNA, 24 bp, Artificial Sequence
  Description of Artificial Sequence Synthetic primer
tcgacttatctgctttttgttaac

SEQ ID NO: 197 - DNA, 23 bp, Artificial Sequence
  Description of Artificial Sequence Synthetic primer
ctatctttgccatcttcatagtc

SEQ ID NO: 198 - DNA, 29 bp, Artificial Sequence
  Description of Artificial Sequence Synthetic primer
gcaataaaaatagaagattctttgtagat

SEQ ID NO: 199 - DNA, 34 bp, Artificial Sequence
  Description of Artificial Sequence Synthetic primer
taaaatttcattttcataaacatcaagattaata

SEQ ID NO: 200 - DNA, 14 bp, Artificial Sequence
  Description of Artificial Sequence Synthetic primer
gcccgacataccca

SEQ ID NO: 201 - DNA, 31 bp, Artificial Sequence
  Description of Artificial Sequence Synthetic primer
ttaatgaaaaagaatacattaagtgcaatat

SEQ ID NO: 202 - DNA, 29 bp, Artificial Sequence
  Description of Artificial Sequence Synthetic primer
ctaataattcataaataaaaaggaggcac

SEQ ID NO: 203 - DNA, 32 bp, Artificial Sequence
  Description of Artificial Sequence Synthetic primer
ttttcaaataaaaaattgaaaaacaaaattgt

SEQ ID NO: 204 - DNA, 34 bp, Artificial Sequence
  Description of Artificial Sequence Synthetic primer
aatatttattcaagatattgaagaatttgaaaaa

SEQ ID NO: 205 - DNA, 35 bp, Artificial Sequence
  Description of Artificial Sequence Synthetic primer
tttaaaatcaaattaagacaatatttttcaaattc

SEQ ID NO: 206 - DNA, 22 bp, Artificial Sequence
  Description of Artificial Sequence Synthetic primer
agcatatttggctttgcttatg

SEQ ID NO: 207 - DNA, 38 bp, Artificial Sequence
  Description of Artificial Sequence Synthetic primer
aaattaaaactttttttattaaagtatacttcatttaa

SEQ ID NO: 208 - DNA, 25 bp, Artificial Sequence
  Description of Artificial Sequence Synthetic primer
gcctgagtattcattatataagtcc

SEQ ID NO: 209 - DNA, 28 bp, Artificial Sequence
  Description of Artificial Sequence Synthetic primer
tatattgggatccaaaatctaatacaag

SEQ ID NO: 210 - DNA, 28 bp, Artificial Sequence
  Description of Artificial Sequence Synthetic primer
caatttctctaattcttcttgcaattag

SEQ ID NO: 211 - DNA, 31 bp, Artificial Sequence
  Description of Artificial Sequence Synthetic primer
ggagtatagtaaggtattacttttgtataaa

SEQ ID NO: 212 - DNA, 32 bp, Artificial Sequence
  Description of Artificial Sequence Synthetic primer
ttcctgagatattcatatttttaatttctttt

SEQ ID NO: 213 - DNA, 19 bp, Artificial Sequence
  Description of Artificial Sequence Synthetic primer
gcaggacttccacttagta

SEQ ID NO: 214 - DNA, 24 bp, Artificial Sequence
  Description of Artificial Sequence Synthetic primer
ggtaggagcttcttttgaataaac

SEQ ID NO: 215 - DNA, 34 bp, Artificial Sequence
  Description of Artificial Sequence Synthetic primer
caaaataggtattttcaaattaaaaatttccata

SEQ ID NO: 216 - DNA, 31 bp, Artificial Sequence
  Description of Artificial Sequence Synthetic primer
aatttaacaattatttgcattccataacata

SEQ ID NO: 217 - DNA, 24 bp, Artificial Sequence
  Description of Artificial Sequence Synthetic primer
gcttagagtctttagatactaggc

SEQ ID NO: 218 - DNA, 22 bp, Artificial Sequence
  Description of Artificial Sequence Synthetic primer
aaagatttcagagctcccatat

SEQ ID NO: 219 - DNA, 29 bp, Artificial Sequence
  Description of Artificial Sequence Synthetic primer
ttctgaaaataaaagagatttttcatctc

SEQ ID NO: 220 - DNA, 28 bp, Artificial Sequence
  Description of Artificial Sequence Synthetic primer
tgactcatgataatttgaaatttgtttg

SEQ ID NO: 221 - DNA, 29 bp, Artificial Sequence
  Description of Artificial Sequence Synthetic primer
aaattatcaggaattttttcaatactgtc

SEQ ID NO: 222 - DNA, 29 bp, Artificial Sequence
  Description of Artificial Sequence Synthetic primer
gcaatacaattttttgtaaaagctaattg

SEQ ID NO: 223 - DNA, 27 bp, Artificial Sequence
  Description of Artificial Sequence Synthetic primer
ccgtaaattttttgagtttcatttgat

SEQ ID NO: 224 - DNA, 22 bp, Artificial Sequence
  Description of Artificial Sequence Synthetic primer
agttacttctggatggaattgt

SEQ ID NO: 225 - DNA, 30 bp, Artificial Sequence
  Description of Artificial Sequence Synthetic primer
aatttttaattatttgatcaccaaattcag

SEQ ID NO: 226 - DNA, 20 bp, Artificial Sequence
  Description of Artificial Sequence Synthetic primer
accgcattagaatccgtaat

SEQ ID NO: 227 - DNA, 19 bp, Artificial Sequence
  Description of Artificial Sequence Synthetic primer
acctctttcacagcaagtt

SEQ ID NO: 228 - DNA, 21 bp, Artificial Sequence
  Description of Artificial Sequence Synthetic primer
catctatagatgacagcaacg

SEQ ID NO: 229 - DNA, 21 bp, Artificial Sequence
  Description of Artificial Sequence Synthetic primer
ttaccaatagctttagcagca

SEQ ID NO: 230 - DNA, 25 bp, Artificial Sequence
  Description of Artificial Sequence Synthetic primer
gtatccaaaccattattttggtgta

SEQ ID NO: 231 - DNA, 25 bp, Artificial Sequence
  Description of Artificial Sequence Synthetic primer
gctaacaatgatccattgtgattat

SEQ ID NO: 232 - DNA, 24 bp, Artificial Sequence
  Description of Artificial Sequence Synthetic primer
tattagggttgatattgcataagc

SEQ ID NO: 233 - DNA, 28 bp, Artificial Sequence
  Description of Artificial Sequence Synthetic primer
cttcatttttcaatccatctaatttttg

SEQ ID NO: 234 - DNA, 21 bp, Artificial Sequence
  Description of Artificial Sequence Synthetic primer
ttagccgcatcaattttttcc

SEQ ID NO: 235 - DNA, 32 bp, Artificial Sequence
  Description of Artificial Sequence Synthetic primer
tcttttaatttattagtaaatgtttcagaaca

SEQ ID NO: 236 - DNA, 22 bp, Artificial Sequence
  Description of Artificial Sequence Synthetic primer
cttctttaccaagatctgtgtg

SEQ ID NO: 237 - DNA, 19 bp, Artificial Sequence
  Description of Artificial Sequence Synthetic primer
cttttgcatcagcatcagt

SEQ ID NO: 238 - DNA, 31 bp, Artificial Sequence
  Description of Artificial Sequence Synthetic primer
tttagttttagtaccatttgtttttaaaatg

SEQ ID NO: 239 - DNA, 28 bp, Artificial Sequence
  Description of Artificial Sequence Synthetic primer
gattcaaataattttccaagttcttcag

SEQ ID NO: 240 - DNA, 19 bp, Artificial Sequence
  Description of Artificial Sequence Synthetic primer
ctgcttttgacaagacctc

SEQ ID NO: 241 - DNA, 24 bp, Artificial Sequence
  Description of Artificial Sequence Synthetic primer
tttaactgaattagcaagcatctc

SEQ ID NO: 242 - DNA, 15 bp, Artificial Sequence
  Description of Artificial Sequence Synthetic primer
ccacaacagggcttg

SEQ ID NO: 243 - DNA, 27 bp, Artificial Sequence
  Description of Artificial Sequence Synthetic primer
gatcttaattaaggtttttttggactt

SEQ ID NO: 244 - DNA, 33 bp, Artificial Sequence
  Description of Artificial Sequence Synthetic primer
ccagttacttttttaaaacaaattaatcttata

SEQ ID NO: 245 - DNA, 29 bp, Artificial Sequence
  Description of Artificial Sequence Synthetic primer
agaaatctttcttgacttatattgacttt

SEQ ID NO: 246 - DNA, 39 bp, Artificial Sequence
  Description of Artificial Sequence Synthetic primer
gaattttaagaaattttttgagaaaataaaaaaataaaa

SEQ ID NO: 247 - DNA, 27 bp, Artificial Sequence
  Description of Artificial Sequence Synthetic primer
tattctttaagagaagagcttaaagtt

SEQ ID NO: 248 - DNA, 30 bp, Artificial Sequence
  Description of Artificial Sequence Synthetic primer
aaattcaatttattaacggcttttgtaata

SEQ ID NO: 249 - DNA, 27 bp, Artificial Sequence
  Description of Artificial Sequence Synthetic primer
tctagcacccaattttgtttatattta

SEQ ID NO: 250 - DNA, 29 bp, Artificial Sequence
  Description of Artificial Sequence Synthetic primer
gtttaagcctacttaaagtctttaaaatc

SEQ ID NO: 251 - DNA, 20 bp, Artificial Sequence
  Description of Artificial Sequence Synthetic primer
cccacactctctctttcaaa

SEQ ID NO: 252 - DNA, 23 bp, Artificial Sequence
  Description of Artificial Sequence Synthetic primer
gatattaaccggcatttaacctt

SEQ ID NO: 253 - DNA, 26 bp, Artificial Sequence
  Description of Artificial Sequence Synthetic primer
tctagcttacaatcccatttataaga

SEQ ID NO: 254 - DNA, 31 bp, Artificial Sequence
  Description of Artificial Sequence Synthetic primer
ccttcaaattttaattttcctctaaaagtta

SEQ ID NO: 255 - DNA, 26 bp, Artificial Sequence
  Description of Artificial Sequence Synthetic primer
ccttcaaaagaagaatcaagatacaa

SEQ ID NO: 256 - DNA, 20 bp, Artificial Sequence
  Description of Artificial Sequence Synthetic primer
cacacccccttttgaagata

SEQ ID NO: 257 - DNA, 27 bp, Artificial Sequence
  Description of Artificial Sequence Synthetic primer
gtaataaccttactattcttgccaata

SEQ ID NO: 258 - DNA, 30 bp, Artificial Sequence
  Description of Artificial Sequence Synthetic primer
ttctactattaatgtatcacaaattaccac

SEQ ID NO: 259 - DNA, 20 bp, Artificial Sequence
  Description of Artificial Sequence Synthetic primer
gcatttacattgcccttcaa

SEQ ID NO: 260 - DNA, 23 bp, Artificial Sequence
  Description of Artificial Sequence Synthetic primer
caaccgctgtttaaataaacctt

SEQ ID NO: 261 - DNA, 30 bp, Artificial Sequence
  Description of Artificial Sequence Synthetic primer
aatattttttttgtttttacatccccatat

SEQ ID NO: 262 - DNA, 30 bp, Artificial Sequence
  Description of Artificial Sequence Synthetic primer
cacacttaccatcaaaaattatattatcat

SEQ ID NO: 263 - DNA, 33 bp, Artificial Sequence
  Description of Artificial Sequence Synthetic primer
aagaaaataaatctacaatttcattagacttta

SEQ ID NO: 264 - DNA, 26 bp, Artificial Sequence
  Description of Artificial Sequence Synthetic primer
caaagtatcttttatttgtgaaacgg

SEQ ID NO: 265 - DNA, 34 bp, Artificial Sequence
  Description of Artificial Sequence Synthetic primer
tctacttattattaattaataaaaaacactgacc

SEQ ID NO: 266 - DNA, 33 bp, Artificial Sequence
  Description of Artificial Sequence Synthetic primer
ctctacgaattaaatttttaagaaaggatttta

SEQ ID NO: 267 - DNA, 26 bp, Artificial Sequence
  Description of Artificial Sequence Synthetic primer
atcaaatccaccattttttttatcca

SEQ ID NO: 268 - DNA, 18 bp, Artificial Sequence
  Description of Artificial Sequence Synthetic primer
ccaaccgccttatttcac

SEQ ID NO: 269 - DNA, 33 bp, Artificial Sequence
  Description of Artificial Sequence Synthetic primer
ttttcaaattatcttcaatcttaaactctttag

SEQ ID NO: 270 - DNA, 25 bp, Artificial Sequence
  Description of Artificial Sequence Synthetic primer
ttttagcaacaactttaaccacttt

SEQ ID NO: 271 - DNA, 17 bp, Artificial Sequence
  Description of Artificial Sequence Synthetic primer
tgtcacgctagatgcag

SEQ ID NO: 272 - DNA, 21 bp, Artificial Sequence
  Description of Artificial Sequence Synthetic primer
ctttacgccacttaaatctgc

SEQ ID NO: 273 - DNA, 24 bp, Artificial Sequence
  Description of Artificial Sequence Synthetic primer
aatcagaaaatattaccccgtttg

SEQ ID NO: 274 - DNA, 31 bp, Artificial Sequence
  Description of Artificial Sequence Synthetic primer
atattattttctaaacctgaagaaggaatat

SEQ ID NO: 275 - DNA, 31 bp, Artificial Sequence
  Description of Artificial Sequence Synthetic primer
cattaaaaaatttgatgatattactttgctc

SEQ ID NO: 276 - DNA, 27 bp, Artificial Sequence
  Description of Artificial Sequence Synthetic primer
gttttgctgttaaagtaaggaaattag

SEQ ID NO: 277 - DNA, 22 bp, Artificial Sequence
  Description of Artificial Sequence Synthetic primer
gctgctagaaaaaaatctcgtt

SEQ ID NO: 278 - DNA, 25 bp, Artificial Sequence
  Description of Artificial Sequence Synthetic primer
ctgctagaaagcgaataattcataa

SEQ ID NO: 279 - DNA, 31 bp, Artificial Sequence
  Description of Artificial Sequence Synthetic primer
gaattttttaaatttgttgcaaaaaaactag

SEQ ID NO: 280 - DNA, 18 bp, Artificial Sequence
  Description of Artificial Sequence Synthetic primer
gcgggtaagaaagacgaa

SEQ ID NO: 281 - DNA, 22 bp, Artificial Sequence
  Description of Artificial Sequence Synthetic primer
gaaaaacgctgtatcaacatga

SEQ ID NO: 282 - DNA, 33 bp, Artificial Sequence
  Description of Artificial Sequence Synthetic primer
attagaaatgtaagtgtaaaaagtgaattaaaa

SEQ ID NO: 283 - DNA, 23 bp, Artificial Sequence
  Description of Artificial Sequence Synthetic primer
cgctctcgtcaaaatttaaaaag

SEQ ID NO: 284 - DNA, 21 bp, Artificial Sequence
  Description of Artificial Sequence Synthetic primer
cttgagaaaaaatgcatctgc

SEQ ID NO: 285 - DNA, 38 bp, Artificial Sequence
  Description of Artificial Sequence Synthetic primer
gatatattaaagctattgtttaataatattattaagga

SEQ ID NO: 286 - DNA, 34 bp, Artificial Sequence
  Description of Artificial Sequence Synthetic primer
attaacttaaatctttgattgactatatttgaat

SEQ ID NO: 287 - DNA, 32 bp, Artificial Sequence
  Description of Artificial Sequence Synthetic primer
aggtttttgaatatattaatcaaaactattgt

SEQ ID NO: 288 - DNA, 31 bp, Artificial Sequence
  Description of Artificial Sequence Synthetic primer
attttgaataaaaaaatttcttattccatgc

SEQ ID NO: 289 - DNA, 26 bp, Artificial Sequence
  Description of Artificial Sequence Synthetic primer
caaagaaaatcatcagacaaaaaagg

SEQ ID NO: 290 - DNA, 35 bp, Artificial Sequence
  Description of Artificial Sequence Synthetic primer
gaatttgaatttaacaataaaaattatttatgctt

SEQ ID NO: 291 - DNA, 29 bp, Artificial Sequence
  Description of Artificial Sequence Synthetic primer
gaattttttgaaaaaatttttattgccag

SEQ ID NO: 292 - DNA, 30 bp, Artificial Sequence
  Description of Artificial Sequence Synthetic primer
ctgtgaaagaaaaatttttaaaagtgaaat

SEQ ID NO: 293 - DNA, 32 bp, Artificial Sequence
  Description of Artificial Sequence Synthetic primer
caatatagtgttattttatgagtttaggaaag

SEQ ID NO: 294 - DNA, 21 bp, Artificial Sequence
  Description of Artificial Sequence Synthetic primer
ttttgttgggggatttttcag

SEQ ID NO: 295 - DNA, 21 bp, Artificial Sequence
  Description of Artificial Sequence Synthetic primer
ttttgttgggggatttttcag

SEQ ID NO: 296 - DNA, 42 bp, Artificial Sequence
  Description of Artificial Sequence Synthetic primer
gcaatatatttatttttttatttatttgttttattgatatta

SEQ ID NO: 297 - DNA, 34 bp, Artificial Sequence
  Description of Artificial Sequence Synthetic primer
gtattatgattgctttatttgtttattacatttc

SEQ ID NO: 298 - DNA, 36 bp, Artificial Sequence
  Description of Artificial Sequence Synthetic primer
gatattatttatcttgtacttatctttttatgtttt

SEQ ID NO: 299 - DNA, 23 bp, Artificial Sequence
  Description of Artificial Sequence Synthetic primer
gtcccaaaattggaaaattttcc

SEQ ID NO: 300 - DNA, 33 bp, Artificial Sequence
  Description of Artificial Sequence Synthetic primer
tatttaaagagcttaaaattaagagaaaagatc

SEQ ID NO: 301 - DNA, 18 bp, Artificial Sequence
  Description of Artificial Sequence Synthetic primer
cgtgaagctgcaagaaaa

SEQ ID NO: 302 - DNA, 23 bp, Artificial Sequence
  Description of Artificial Sequence Synthetic primer
tggaaaagcaataaaagctgctg

SEQ ID NO: 303 - DNA, 27 bp, Artificial Sequence
  Description of Artificial Sequence Synthetic primer
tgttgtatatgaacatttattggaaat

SEQ ID NO: 304 - DNA, 24 bp, Artificial Sequence
  Description of Artificial Sequence Synthetic primer
gcttggtaattctgagataagaaa

SEQ ID NO: 305 - DNA, 23 bp, Artificial Sequence
  Description of Artificial Sequence Synthetic primer
cctcaatttgaaggtcaaacaaa

SEQ ID NO: 306 - DNA, 23 bp, Artificial Sequence
  Description of Artificial Sequence Synthetic primer
attttaaagaggggcttacagct

SEQ ID NO: 307 - DNA, 21 bp, Artificial Sequence
  Description of Artificial Sequence Synthetic primer
gccatgaatgaagcttttaaa

SEQ ID NO: 308 - DNA, 25 bp, Artificial Sequence
  Description of Artificial Sequence Synthetic primer
ctcatgttatgggatttagaagtgg

SEQ ID NO: 309 - DNA, 25 bp, Artificial Sequence
  Description of Artificial Sequence Synthetic primer
ctgacaacattctttcttttgttaa

SEQ ID NO: 310 - DNA, 21 bp, Artificial Sequence
  Description of Artificial Sequence Synthetic primer
tgttaatgtggggcttaaatg

SEQ ID NO: 311 - DNA, 22 bp, Artificial Sequence
  Description of Artificial Sequence Synthetic primer
gcttttcaatcagaaccttatt

SEQ ID NO: 312 - DNA, 22 bp, Artificial Sequence
  Description of Artificial Sequence Synthetic primer
gagggtgggataaaatcttttt

SEQ ID NO: 313 - DNA, 28 bp, Artificial Sequence
  Description of Artificial Sequence Synthetic primer
tggtaaagaaaaatcttcaaaattttat

SEQ ID NO: 314 - DNA, 30 bp, Artificial Sequence
  Description of Artificial Sequence Synthetic primer
cgataaaatatacatttcaattgaagataa

SEQ ID NO: 315 - DNA, 20 bp, Artificial Sequence
  Description of Artificial Sequence Synthetic primer
ggcttaaagagcttgctttt

SEQ ID NO: 316 - DNA, 28 bp, Artificial Sequence
  Description of Artificial Sequence Synthetic primer
agattataatttcgatgttcttgaaaaa

SEQ ID NO: 317 - DNA, 25 bp, Artificial Sequence
  Description of Artificial Sequence Synthetic primer
cggattctgaaatttttgaaacttt

SEQ ID NO: 318 - DNA, 21 bp, Artificial Sequence
  Description of Artificial Sequence Synthetic primer
ggggactaaggttactttttt

SEQ ID NO: 319 - DNA, 25 bp, Artificial Sequence
  Description of Artificial Sequence Synthetic primer
agaagttgtgggggaatcttctgtt

SEQ ID NO: 320 - DNA, 24 bp, Artificial Sequence
  Description of Artificial Sequence Synthetic primer
ctttttcaaaaggtattccgactt

SEQ ID NO: 321 - DNA, 27 bp, Artificial Sequence
  Description of Artificial Sequence Synthetic primer
ggtttatgttaatagagatggaaaaat

SEQ ID NO: 322 - DNA, 24 bp, Artificial Sequence
  Description of Artificial Sequence Synthetic primer
ggttgtaaatgctctatcttcgtt

SEQ ID NO: 323 - DNA, 25 bp, Artificial Sequence
  Description of Artificial Sequence Synthetic primer
tggtaagtttaataaaggcacgtat

SEQ ID NO: 324 - DNA, 26 bp, Artificial Sequence
  Description of Artificial Sequence Synthetic primer
ccttgaacttgttttaacaaaattac

SEQ ID NO: 325 - DNA, 21 bp, Artificial Sequence
  Description of Artificial Sequence Synthetic primer
accgatattcatgaagaggag

SEQ ID NO: 326 - DNA, 23 bp, Artificial Sequence
  Description of Artificial Sequence Synthetic primer
tacccattttagcacttcctcca

SEQ ID NO: 327 - DNA, 20 bp, Artificial Sequence
  Description of Artificial Sequence Synthetic primer
tggcaaaatggcctgaaaaa

SEQ ID NO: 328 - DNA, 25 bp, Artificial Sequence
  Description of Artificial Sequence Synthetic primer
ttgttttctcaacattaagcatttt

SEQ ID NO: 329 - DNA, 25 bp, Artificial Sequence
  Description of Artificial Sequence Synthetic primer
atcattggtgataaccttatcttct

SEQ ID NO: 330 - DNA, 18 bp, Artificial Sequence
  Description of Artificial Sequence Synthetic primer
actcctgcaccaagagat

SEQ ID NO: 331 - DNA, 24 bp, Artificial Sequence
  Description of Artificial Sequence Synthetic primer
atcttgtgataacgaagttttgta

SEQ ID NO: 332 - DNA, 20 bp, Artificial Sequence
  Description of Artificial Sequence Synthetic primer
tccatcaacatcggcatctg

SEQ ID NO: 333 - DNA, 24 bp, Artificial Sequence
  Description of Artificial Sequence Synthetic primer
aaaagctaaaagcaaagttctaat

SEQ ID NO: 334 - DNA, 29 bp, Artificial Sequence
  Description of Artificial Sequence Synthetic primer
atatatccattttcaattaaatctctcat

SEQ ID NO: 335 - DNA, 20 bp, Artificial Sequence
  Description of Artificial Sequence Synthetic primer
tataaagaggaggcatggct

SEQ ID NO: 336 - DNA, 30 bp, Artificial Sequence
  Description of Artificial Sequence Synthetic primer
taaaaataataaatacgattgtcatacttt

SEQ ID NO: 337 - DNA, 26 bp, Artificial Sequence
  Description of Artificial Sequence Synthetic primer
tattgcgatttttagtttcaatagaa

SEQ ID NO: 338 - DNA, 23 bp, Artificial Sequence
  Description of Artificial Sequence Synthetic primer
cccaagccctttatatctctgaa

SEQ ID NO: 339 - DNA, 18 bp, Artificial Sequence
  Description of Artificial Sequence Synthetic primer
agctgcgttggattcatc

SEQ ID NO: 340 - DNA, 22 bp, Artificial Sequence
  Description of Artificial Sequence Synthetic primer
ctagcaggatccatagttgttt

SEQ ID NO: 341 - DNA, 28 bp, Artificial Sequence
  Description of Artificial Sequence Synthetic primer
atcatctatattcatcaatctcattttt

SEQ ID NO: 342 - DNA, 26 bp, Artificial Sequence
  Description of Artificial Sequence Synthetic primer
gagtaacaaaaattttttcagcttca

SEQ ID NO: 343 - DNA, 21 bp, Artificial Sequence
  Description of Artificial Sequence Synthetic primer
tttctgggctcaactaaatct

SEQ ID NO: 344 - DNA, 28 bp, Artificial Sequence
  Description of Artificial Sequence Synthetic primer
gattaattacattaagtgcattctgttc

SEQ ID NO: 345 - DNA, 21 bp, Artificial Sequence
  Description of Artificial Sequence Synthetic primer
ccattaacgctccaattacac

SEQ ID NO: 346 - DNA, 31 bp, Artificial Sequence
  Description of Artificial Sequence Synthetic primer
tcctaacatttaatatttgttctttattttc

SEQ ID NO: 347 - DNA, 33 bp, Artificial Sequence
  Description of Artificial Sequence Synthetic primer
gcataatttaaataagaagtttttatttcatct

SEQ ID NO: 348 - DNA, 25 bp, Artificial Sequence
  Description of Artificial Sequence Synthetic primer
gaagagctctagaaacaataactga

SEQ ID NO: 349 - DNA, 23 bp, Artificial Sequence
  Description of Artificial Sequence Synthetic primer
tggtttaagaccatctcttacgt

SEQ ID NO: 350 - DNA, 28 bp, Artificial Sequence
  Description of Artificial Sequence Synthetic primer
ctcatacatagaataaagtattctcctg

(19) United States
(12) Patent Application Publication
(10) Pub. No.: US 2015/0031038 A1
(43) Pub. Date: Jan. 29, 2015

(54) SAMPLE PREPARATION METHODS

(71) Applicant: Ibis Biosciences, Inc., Carlsbad, CA (US)

(72) Inventors: Mark W. Eshoo, San Diego, CA (US); Christopher D. Crowder, San Marcos, CA (US)

(21) Appl. No.: 14/342,284

(22) PCT Filed: Sep. 6, 2012
(86) PCT No.: PCT/US12/53909

§ 371 Date: Sep. 15, 2014

Related U.S. Application Data
(60) Provisional application No. 61/531,471, filed on Sep. 6, 2011.

(51) Int. Cl. C12Q 1/68

(52) U.S. Cl. CPC C12Q 1/6806
USPC 435/6.12; 435/6.1

(57) ABSTRACT

The present invention provides whole blood nucleic acid extraction methods, compositions, and kits, as well as nested isothermal amplification methods, compositions, and kits. In certain embodiments, these methods are applied to detecting Lyme disease, including in patients without classic erythema migrans skin lesions.

### CROSS-REFERENCE TO RELATED APPLICATIONS

The present Application claims priority to U.S. Provisional Application Ser. No. 61/531,471 filed Sep. 6, 2011, the entirety of which is incorporated by reference herein.

### STATEMENT REGARDING FEDERALLY SPONSORED RESEARCH OR DEVELOPMENT

This invention was made with government support under 1R43AI077156-01 awarded by the National Institutes of Health. The government has certain rights in the invention.

### FIELD OF THE INVENTION

The present invention provides whole blood nucleic acid extraction methods, compositions, and kits, as well as nested isothermal amplification methods, compositions, and kits. In certain embodiments, these methods are applied to detecting Lyme disease, including in patients without classic erythema migrans skin lesions.

### BACKGROUND

The diagnosis of acute infectious diseases is problematic when the pathogens are not in abundance, not easily cultured or the antibody response to them is delayed. Lyme disease is such an example. Infection by the causative agent, Borrelia burgdorferi^{1,2}, is often difficult to diagnose because of clinical variability, including variations of the erythema migrans (EM) skin lesion and the lack of high performing diagnostic tests. A strategy that results in early unambiguous diagnosis of the infection will favorably impact management of the patient.

There are currently clinical limitations to early diagnosis. The best clinical marker of acute infection, the skin lesion erythema migrans (EM), is absent in at least 30% of the cases.³ Even when present, the EM is often not the classic bull's-eye lesion but rather an uncharacteristic variant as reported in 25-30% of cases.^{4,5} This may require referral to a specialist or prompt further diagnostic testing, thereby delaying the start of antibiotic therapy at the time for the best chance of cure without sequelae.

These clinical hurdles to diagnosis are compounded by the limitations of current laboratory tests. The most commonly used assays are serologic. However, these assays have been hampered by the biologically delayed antibody response (often 3 weeks or more to reach detectable levels). They are further limited because a single positive test is an indirect measure of exposure and cannot distinguish active from past infection. A direct Borrelia molecular test such as PCR can have high specificity but typically the tests have lacked sensitivity when applied to blood. In the past, PCR assays for Lyme disease did not have the benefit of current advances in sample preparation.⁶ and enhancement methods.^{7,8} such as Borrelia-targeted DNA enrichment (akin to a “molecular culture”) that may allow detection of a low number of nucleic acid copies in the blood. In addition, the optimal blood volume and component of the sample, such as whole blood versus serum, may have contributed to prior limitations of PCR based diagnostics.

### SUMMARY OF THE INVENTION

The present invention provides whole blood nucleic acid extraction methods, compositions, and kits, as well as nested isothermal amplification methods, compositions, and kits. In certain embodiments, these methods are applied to detecting Lyme disease, including in patients without classic erythema migrans skin lesions.

In some embodiments, the present description provides methods of nucleic acid extraction comprising: a) contacting a sample of whole blood (e.g., EDTA treated whole blood) with beads, a proteinase, and an anionic surfactant (e.g., SDS) to generate a treated sample; b) homogenizing the treated sample to generate a cell lysate; c) centrifuging the cell lysate comprising a supernatant; d) separating the supernatant from the cell lysate; e) adding magnetic particles and lysis buffer to the supernatant to generate a magnetic-particle sample, wherein the magnetic particles are configured to bind nucleic acid molecules; f) washing the magnetic-particle sample with a wash buffer; g) treating the magnetic-particle sample in order to generate a dried magnetic bead sample; and h) treating the dried magnetic bead sample with an elution buffer such that a purified nucleic acid sample is generated that comprises purified nucleic acid.

In certain embodiments, the methods further comprise subjecting the purified nucleic acid to PCR and/or isothermal nested PCR to generate amplified nucleic acid. In some embodiments, the methods further comprise subjecting the amplified nucleic acid to mass spectrometry bioagent analysis in order to identify the source of the purified nucleic acid. In other embodiments, the mass spectrometry bioagent analysis comprises electrospray ionization mass spectrometry and base composition analysis.

In some embodiments, the present disclosure provides methods comprising: a) contacting a sample comprising isolated nucleic acid with a buffer, dNTPs, and a plurality of nested PCR primer pairs configured to amplify at least part of at least one bioagent target sequence; b) incubating the sample with a DNA polymerase under isothermal conditions such that amplified nucleic acid is generated; c) inactivating the DNA polymerase; and d) subjecting the amplified nucleic acid to mass spectrometry bioagent analysis in order to identify the source of the isolated nucleic acid.

In other embodiments, the mass spectrometry bioagent analysis comprises electrospray ionization mass spectrometry and base composition analysis. In certain embodiments, the DNA polymerase comprises BstE DNA polymerase. In additional embodiments, the incubating is conducted at about 56 degrees Celsius. In further embodiments, inactivating the DNA polymerase comprises heating the sample to at least about 80 degrees Celsius. In other embodiments, the plurality of nested PCR primer pairs comprises at least 10 primer pairs (e.g., 10 . . . 15 . . . 19, etc). In particular embodiments, the plurality of nested PCR primer pairs comprises at least 20 primer pairs (e.g., 20 . . . 25 . . . 35 . . . 45 . . . etc.). In further embodiments, the at least one bioagent target sequence comprises at least five bioagent target sequences. In certain embodiments, the methods further comprise a step after c) but before d) of further amplifying the amplified nucleic acid without any purification of the amplified nucleic acid.

### DESCRIPTION OF THE FIGURES

FIG. 1 shows an atypical EM lesion from a patient who was PCR positive and seronegative with a negative ELISA after eight days of illness. Repeat serology at the end of therapy 3 weeks later showed a positive ELISA, positive IgM western blot and negative IgG western blot.

### DETAILED DESCRIPTION

The present invention provides whole blood nucleic acid extraction methods, compositions, and kits, as well as nested isothermal amplification methods, compositions, and kits. In certain embodiments, these methods are applied to detecting Lyme disease, including in patients without classic erythema migrans skin lesions.

The methods, kits, and compositions are useful with any target nucleic acid sequence and are not limited to any particular target sequence (e.g., not limited to nucleic acid sequences from B. burgdorferi). The discussion below is focused on detecting sequence from B. burgdorferi. Such target sequences are exemplary and are not intended to limit the scope of the present description. Other target sequences (e.g., from infections disease) are well known in the art. Also well know in the art are primers, including nested prior sets, that are useful in the present description.

Lyme disease is representative of an infectious disease where early diagnosis is imperative to avoid sequelae. However diagnosis is often difficult because the clinical manifestations, including the rash, are variable and the pathogens are often not in abundance, not easily cultured, and the antibody response to them is delayed. In the past PCR for B. burgdorferi in blood had low sensitivity. This may be related to low copy number, insufficient volume of blood or targeting the wrong component of blood

The present description overcomes some of these obstacles by combining a pre-PCR nucleic acid enrichment with sensitive PCR detection from nucleic acid extraction from 1.25 ml of whole blood from patients with skin lesions and early Lyme disease. With this strategy, PCR positivity was found in 14 of 29 (p=0·0001) endemic area subjects with typical erythema migrans (EM) or a variant, often before seroconversion was positive. The enrichment technique increased the yield from 2 to 14. None of 44 control subjects were positive. A serendipitous and unexpected finding of clinical importance was the observation that 8 of 14 (57%) of PCR positive subjects had an atypical EM.

The description provides improved early diagnosis of Lyme disease by combination of a pre-PCR Borrelia DNA enhancement, sensitive PCR, and targeting sufficient volumes of whole blood. The surprise finding of non-classic EM lesions in the majority of microbiologically proven Lyme disease cases serves as alert to clinicians evaluating patients with endemic area exposure.

In certain embodiments, the PCR generated amplicons are detected by mass spectrometetry methods using bioagent identifying amplicons. In some embodiments, primers are selected to hybridize to conserved sequence regions of nucleic acids derived from a bioagent and which flank variable sequence regions to yield a bioagent identifying amplicon which can be amplified and which is amenable to base composition analysis. In some embodiments, the corresponding base composition of one or more different amplicons is queried against a database of base compositions indexed to bioagents and to the primer pair used to generate the amplicon. A match of the measured base composition to a database entry base composition associates the sample bioagent to an indexed bioagent in the database. Thus, the identity of the unknown bioagent is determined. No prior knowledge of the unknown bioagent is necessary to make an identification. In some instances, the measured base composition associates with more than one database entry base composition. Thus, a second/subsequent primer pair is generally used to generate an amplicon, and its measured base composition is similarly compared to the database to determine its identity in triangulation identification. Furthermore, the methods and other aspects of the invention can be applied to rapid parallel multiplex analyses, the results of which can be employed in a triangulation identification strategy. Thus, in some embodiments, the present invention provides rapid throughput and does not require nucleic acid sequencing or knowledge of the linear sequences of nucleobases of the amplified target sequence for bioagent detection and identification.

Methods of employing base compositions, databases containing base composition entries, and triangulation using primers, are described in the following patents, patent applications and scientific publications, all of which are herein incorporated by reference as if fully set forth herein: U.S. Pat. Nos. 7,108,974; 7,217,510; 7,226,739; 7,255,992; 7,312,036; 7,339,051; US patent publication numbers 2003/0027135; 2003/0167133; 2003/0167134; 2003/0175695; 2003/0175696; 2003/0175697; 2003/0187588; 2003/0187593; 2003/0190605; 2003/0225529; 2003/0228571; 2004/0110169; 2004/0117129; 2004/0121309; 2004/0121310; 2004/0121311; 2004/0121312; 2004/0121313; 2004/0121314; 2004/0121315; 2004/0121329; 2004/0121335; 2004/0121340; 2004/0122598; 2004/0122857; 2004/0161770; 2004/0185438; 2004/0202997; 2004/0209260; 2004/0219517; 2004/0253583; 2004/0253619; 2005/0027459; 2005/0123952; 2005/0130196 2005/0142581; 2005/0164215; 2005/0266397; 2005/0270191; 2006/0014154; 2006/0121520; 2006/0205040; 2006/0240412; 2006/0259249; 2006/0275749; 2006/0275788; 2007/0087336; 2007/0087337; 2007/0087338 2007/0087339; 2007/0087340; 2007/0087341; 2007/0184434; 2007/0218467; 2007/0218467; 2007/0218489; 2007/0224614; 2007/0238116; 2007/0243544; 2007/0248969; WO2002/070664; WO2003/001976; WO2003/100035; WO2004/009849; WO2004/052175; WO2004/053076; WO2004/053141; WO2004/053164; WO2004/060278; WO2004/093644; WO 2004/101809; WO2004/111187; WO2005/023083; WO2005/023986; WO2005/024046; WO2005/033271; WO2005/036369; WO2005/086634; WO2005/089128; WO2005/091971; WO2005/092059; WO2005/094421; WO2005/098047; WO2005/116263; WO2005/117270; WO2006/019784; WO2006/034294; WO2006/071241; WO2006/094238; WO2006/116127; WO2006/135400; WO2007/014045; WO2007/047778; WO2007/086904; WO2007/100397; and WO2007/118222, all of which are herein incorporated by reference.

Exemplary base-count related methods and other aspects of use in the methods, systems, and other aspects of the invention are also described in, for example, Ecker et al., Ibis T5000: a universal biosensor approach for microbiology. Nat Rev Microbiol. 2008 Jun. 3.; Ecker et al., The Microbial Rosetta Stone Database: A compilation of global and emerging infectious microorganisms and bioterrorist threat agents.; Ecker et al., The Ibis T5000 Universal Biosensor: An Automated Platform for Pathogen Identification and Strain Typing.; Ecker et al., The Microbial Rosetta Stone Database: A common structure for microbial biosecurity threat agents.; Ecker et al., Identification of Acinetobacter species and genotyping of Acinetobacter baumannii by multilocus PCR and mass spectrometry. J Clin Microbiol. 2006 August; 44(8):2921-32.; Ecker et al., Rapid identification and strain-typing of respiratory pathogens for epidemic surveillance. Proc Natl Acad Sci USA. 2005 May 31; 102(22):8012-7. Epub 2005 May 23.; Wortmann et al., Genotypic evolution of Acinetobacter baumannii Strains in an outbreak associated with war trauma, Infect Control Hosp Epidemiol. 2008 June; 29(6):553-555.; Hannis et al., High-resolution genotyping of Campylobacter species by use of PCR and high-throughput mass spectrometry. J Clin Microbiol. 2008 April; 46(4): 1220-5.; Blyn et al., Rapid detection and molecular serotyping of adenovirus by use of PCR followed by electrospray ionization mass spectrometry. J Clin Microbiol. 2008 February; 46(2):644-51.; Eshoo et al., Direct broad-range detection of alphaviruses in mosquito extracts, Virology. 2007 Nov. 25; 368(2):286-95.; Sampath et al., Global surveillance of emerging Influenza virus genotypes by mass spectrometry. PLoS ONE. 2007 May 30; 2(5):e489.; Sampath et al., Rapid identification of emerging infectious agents using PCR and electrospray ionization mass spectrometry. Ann N Y Acad Sci. 2007 April; 1102: 109-20.; Hujer et al., Analysis of antibiotic resistance genes in multidrug-resistant Acinetobacter sp. isolates from military and civilian patients treated at the Walter Reed Army Medical Center. Antimicrob Agents Chemother. 2006 December; 50(12):4114-23.; Hall et al., Base composition analysis of human mitochondrial DNA using electrospray ionization mass spectrometry: a novel tool for the identification and differentiation of humans. Anal Biochem. 2005 Sep. 1; 344(1):53-69.; Sampath et al., Rapid identification of emerging pathogens: coronavirus. Emerg Infect Dis. 2005 March; 11(3):373-9.; Jiang Y, Hofstadler S A. A highly efficient and automated method of purifying and desalting PCR products for analysis by electrospray ionization mass spectrometry; Jiang et al., Mitochondrial DNA mutation detection by electrospray mass spectrometry; Russell et al., Transmission dynamics and prospective environmental sampling of adenovirus in a military recruit setting; Hofstadler et al., Detection of microbial agents using broad-range PCR with detection by mass spectrometry: The TIGER concept. Chapter in; Hofstadler et al., Selective ion filtering by digital thresholding: A method to unwind complex ESI-mass spectra and eliminate signals from low molecular weight chemical noise.; Hofstadler et al., TIGER: The Universal Biosensor.; Van Ert et al., Mass spectrometry provides accurate characterization of two genetic marker types in Bacillus anthracis.; Sampath et al., Forum on Microbial Threats: Learning from SARS: Preparing for the Next Disease Outbreak—Workshop Summary (ed. Knobler S E, Mahmoud A, Lemon S.) The National Academies Press, Washington, D.C. 2004. 181-185.

### EXAMPLES

### Examples 1

### Early Lyme Patient Specimens.

The current Example is part of a larger, ongoing longitudinal cohort study of early Lyme disease being conducted in a suburban community of a medium-sized, Northeast city since the summer of 2008. Adult patients with early, untreated Lyme disease are referred to a primary care physician with infectious disease training (JA) and provide written consent to participate.

Eligible patients are required to be treatment naïve, to have a documented rash diagnosed as EM at time of enrollment, and to have evidence of systemic infection; typically manifest as dissemination of the primary EM lesion or concurrent onset of new flu-like or other symptoms. Patients with a prior history of Lyme disease or symptom duration of their current illness of longer than 3 months are excluded.²⁰. Forty four negative control specimens were obtained from Biomed Supply Inc. (Carlsbad, Calif.). Specimens were collected at a donation site in Pennsylvania from healthy donors screened by Biomed Supply Inc. A paired 7 mL tube of EDTA treated whole blood and 5-12 mL of serum was provided for each control patient.

### Serological and Other Analyses.

At the initial, pre-treatment study visit, self-report demographics, medical history information, complete blood counts (CBC), complete metabolic panels (CMP) and 2-tier antibody testing for B. burgdorferi were performed as part of the patient clinical evaluation. All serologic testing for both the patient specimens and the negative controls was performed through the same commercial laboratory, and results were interpreted according to CDC criteria¹¹. Based on these criteria, approximate illness duration was documented and included in determining serostatus; patients with less than 4 weeks illness duration were positive based on either IgM or IgG positivity on western blot analysis, whereas patients with greater than 4 weeks illness duration were required to be IgG positive. Photos of EM rashes were reviewed by a panel of experienced clinicians without knowledge of serologic or PCR status. Skin lesions with classic bull's eye appearance with central clearing and peripheral erythema were classified as classic EM lesions. EM with uniform red or red-blue appearance which lacked central clearing were classified as non-classic EM lesions.

DNA Extraction from Blood Specimens:

A combination of bead-beating and magnetic bead isolation was used to extract nucleic acids from 1.25 mL of whole EDTA blood. The blood was mixed in 2.0 mL screw-cap tubes (Sarstedt, Newton N.C.) filled with 1.35 g of 0.1-mm yttria-stabilized zirconium oxide beads (Glen Mills, Clifton, N.J.), 25 μL proteinase K solution (Qiagen, Valencia, Calif.), 142 μL of 20% SDS solution (Ambion, Austin, Tex.) and 1 uL of DNA extraction control (Abbott Molecular, Des Plains, Ill.). The mixture was then homogenized in a Precellys 24 tissue homogenizer (Bioamerica Inc., Miami, Fla.) at 6,200 rpm for 3 sets of 90 sec with 5 sec between sets. The homogenized lysates were then incubated at 56° C. for 15 min and then centrifuged for 3 min at 16,000 g in a bench top microcentrifuge. To isolate nucleic acids 1 mL of the supernatant was transferred to a 24-well deep-well Kingfisher plate (Thermo Scientific, Waltham, Mass.) along with 1.1 mL of Abbott lysis buffer (Abbott Molecular), and 160 μL of magnetic particles (Abbott Molecular). The specimens were incubated for 16.5 minutes in the lysis buffer at 56° C. Specimens were then washed once in Wash buffer I (Abbott Molecular), and three times in Wash buffer II (Abbott Molecular), with 1 min incubation for each wash step. The magnetic beads were then dried for 3 min at 65° C., and nucleic acids were eluted into 250 μL of elution buffer (Abbott Molecular), by incubating the magnetic particles at 65° C. for 3 min.

Nested Isothermal Amplification of B. burgdorferi Target DNA.

To increase sensitivity of B. burgdorferi detection by PCR/ESI-MS the seven target regions (eight primer pairs) of the previously described Borrelia genotyping assay²¹ were enriched by a nested isothermal amplification. For each of the seven target regions were amplified using 50 oligonucleotide primers: 25 upstream and 25 downstream to the DNA region of interest (Table 1).

TABLE 1

| Primer Pair | Gene | | |
|---|---|---|---|
| Target | Target | Primer | Sequence 3′-5′ |
| BCT3514 | rpoC | 3514E3-F1 | CCGAAAAAGATGGGCTTTT |
| | | 3514E3-F2 | AGGTTAAAAAGTCCGAAACTATT |
| | | 3514E3-F3 | TCTCCCGATCAAATTAGAAATTG |
| | | 3514E3-F4 | AAAGAGATAAAAGATTTTGAAAGAATAAA |
| | | 3514E3-F5 | AAAGCTAGGTTTTTGGAGTTTT |
| | | 3514E3-F6 | ACAGAAAAAGAAGAAGAATTGATTAA |
| | | 3514E3-F7 | ATGATGCTGGGAATCAGGTTC |
| | | 3514E3-F8 | GGGCTTGGACTTGATTTG |
| | | 3514E3-F9 | GTCTTTTAATGTGCTAATGCAAGA |
| | | 3514E3-F10 | GGTTCCTACTAATGTATCAGGG |
| | | 3514E3-F11 | GGCAGAGTTAAAATATATGAAAATATAG |
| | | 3514E3-F12 | CACCCTTCAAGAACTTTTAACAG |
| | | 3514E3-F13 | GGCTCTTGAAGCTTATGG |
| | | 3514E3-F14 | AGACTTGGAGAAATGGAGG |
| | | 3514E3-F15 | GAGGAAAGGCTCAATTTGG |
| | | 3514E3-F16 | TCTTGTTTCTCAGCAACCT |
| | | 3514E3-F17 | CGCAAGATCAACAGGC |
| | | 3514E3-P18 | ACTACACCATCTTGTTGATGATA |
| | | 3514E3-F19 | GTAATGGTTGGGGTGATTTAC |
| | | 3514E3-F20 | GGAGAGCCGTTCGAAA |
| | | 3514E3-F21 | CCAACTTCTAAAGAAATTTTATATGATGG |
| | | 3514E3-F22 | AGGAAAAATTAAAAACTGCTGGA |
| | | 3514E3-F23 | TGTTTTTGAATCTGCTACAAATGA |
| | | 3514E3-F24 | CTGGTAAATATCTTGGTGAATCTTATAA |
| | | 3514E3-F25 | GGACAGTTAATGGAATCTCAAT |
| | | 3514E3-R1 | ATACCAAATATGAGCAACTGGGGC |
| | | 3514E3-R2 | AAGCCCAATCCTAGAGGGTA |
| | | 3514E3-R3 | TAGAATTCAAACTAGATGCTGTAAT |
| | | 3514E3-R4 | GCCTTCAATTACTACATATTTTTCATA |
| | | 3514E3-R5 | GGCCGGTTCAATTACTACA |
| | | 3514E3-R6 | TCTTCATTTAAAAGCTGCATTTTT |
| | | 3514E3-R7 | GCTCTCTAGCTTCTATGTACTCA |
| | | 3514E3-R8 | AAGCATTAAAAGACATACCATATCGC |
| | | 3514E3-R9 | GAAGAGTTTTAATAGCCTCAGCCC |
| | | 3514E3-R10 | GACGAAAGCTCATCAAGATCA |
| | | 3514E3-R11 | CAGTTTTATCATCTTTATCTATCATTTGAA |
| | | 3514E3-R12 | AAATTCTCAATAATTTCAAGACGTCTT |
| | | 3514E3-R13 | ATCCACTCTGGCTTATTGCCA |
| | | 3514E3-R14 | GGGGAATAACAGGAAGAAC |
| | | 3514E3-R15 | GCTGAACCATTGGCC |
| | | 3514E3-R16 | ATGTTGCAAAGCGCC |
| | | 3514E3-R17 | CGATTATTTCTATTTATGACTCTTCTATAAAGATC |
| | | 3514E3-R18 | GCATTAAGAAGAAGCAACTTTCT |
| | | 3514E3-R19 | ATTCTTTTTTCGTTTCTCACAATAATC |
| | | 3514E3-R20 | GTCAAAAAGAGAGTCTACTGATTC |
| | | 3514E3-R21 | GAACCTTTGACAACCTTTCTTTTA |
| | | 3514E3-R22 | CGACTTGAGAGGCCT |
| | | 3514E3-R23 | CCTGCTTACCTTTTAATGCAT |
| | | 3514E3-R24 | TTTTACCAAGAAGATTTTGCCTAA |
| | | 3514E3-R25 | CAATAACAGAACGACCAGAATAA |
| BCT3515 | rplB | 3515E3-F1 | GTGTTGCTATGAATCCTGTTG |
| | | 3515E3-F2 | GGTAGAAGACCCAAGGT |
| | | 3515E3-F3 | GGAAAGCTGGTAAAAGTAGG |
| | | 3515E3-F4 | TGGAAATGAAGATTATGCCAATATTT |
| | | 3515E3-F5 | TTTTAAAAAATGTATTGCAACAATTGG |
| | | 3515E3-F6 | TTATCATCTGGCGAGATGAG |
| | | 3515E3-F7 | GACGGGAATTATGTCACTG |
| | | 3515E3-F8 | GGTGGATATGCTATGATACTTG |
| | | 3515E3-F9 | GGGTGGACAGCTTATAAGA |
| | | 3515E3-F10 | CGTTCACAATATTGAGCTTAATGT |
| | | 3515E3-F11 | CTTACCTCTTGAAAATATTCCTATTGG |
| | | 3515E3-F12 | CTAATGCTCCAATTAAAATTGGC |
| | | 3515E3-F13 | GGTTGGAGATGTTTTGGAAAG |
| | | 3515E3-F14 | AAAGGTATATTATTTCTCCTAAAGGC |
| | | 3515E3-F15 | GCTAATATAGCTTTGCTTGTTTATAAAG |
| | | 3515E3-F16 | GTTGCTTCTATTGAATATGATCCTAA |
| | | 3515E3-F17 | CGAAGAGATAAATTTAGCATTCCTG |
| | | 3515E3-F18 | GCATAAGAGAAAGTATAGGTTGATTG |
| | | 3515E3-F19 | CTGGTAGGATTAGTATTAGAAGAAGAG |
| | | 3515E3-F20 | AAAAGGTAAAAAATTTAAATCGGGC |
| | | 3515E3-F21 | CAAAGGTAATGATCCTTTGAAATC |
| | | 3515E3-F22 | GCTATAAGACGACTTTATCTTTTGAT |
| | | 3515E3-F23 | AGACTTATAAGCCAAAAACTTCTTC |
| | | 3515E3-F24 | GGTTTTGGAGAAAAATAAATATGGG |
| | | 3515E3-F25 | CAAAAAGGAAGATAAAATAGATATTTTTTAGTC |
| | | 3515E3-R1 | TTTCTTGCGAGTCTTATAACCT |
| | | 3515E3-R2 | TTTATTTCTTCTTTTAATAATAAATTTATCTGAATATC |
| | | 3515E3-R3 | TTTTTTAATAGATCTTGCCACTATACTC |
| | | 3515E3-R4 | ACTTTTTGATAAAGACTCTTTTCTATAAAAG |
| | | 3515E3-R5 | CTTCTCACTTCCAAAAGACG |
| | | 3515E3-R6 | AAGATCTGGAGTAGGTTTTAATAAC |
| | | 3515E3-R7 | GGCTTACCATTTCAGGAATTAT |
| | | 3515E3-R8 | AAGTTTTGCCATTGTAAACAGATAT |
| | | 3515E3-R9 | CAAGATCCTCGGTAATATAAATAGGT |
| | | 3515E3-R10 | CTCGCCAAGCTTATGTC |
| | | 3515E3-R11 | CCTCTAAAAATCCTTGTAGGTG |
| | | 3515E3-R12 | CCTCTAAAAATCCTTGTAGGTG |
| | | 3515E3-R13 | CTTCCCTTTTTATCTGACTTAGC |
| | | 3515E3-R14 | ATCTTCTATTTACCAACATAACTACTTAC |
| | | 3515E3-R15 | AGGGTAAATTTTTGCCCTTTG |
| | | 3515E3-R16 | CTATTGGCCTAACTTTTTTTGG |
| | | 3515E3-R17 | AGACTCTCCCCGGATATT |
| | | 3515E3-R18 | AAAGCACTGCAATAGCC |
| | | 3515E3-R19 | TAAAAGCTTAGCTCCTTTATTAGG |
| | | 3515E3-R20 | TGATGCTGCTGACTTAACAA |
| | | 3515E3-R21 | CTCGGAAAGATTTTTATTGTGATACA |
| | | 3515E3-R22 | CATCAACCATAACTGTTTTAACAAATATC |
| | | 3515E3-R23 | GCCAAATCTTTTTACGACGAC |
| | | 3515E3-R24 | TATCAGCTCTACCCCTAGC |
| | | 3515E3-R25 | CTTCAACAAAAATATGACAATTTCTATTAAC |
| BCT3516 | leuS | 3516E3-F1 | CCTATCCTTCTGCCAACG |
| | | 3516E3-F2 | GGAAAAAAGATTGTATATACTTGACATG |
| | | 3516E3-F3 | CAAAGTAGAAGAAGATCCAAGTATTC |
| | | 3516E3-F4 | GGCAAGAATTTTGGGATAATAACA |
| | | 3516E3-F5 | TGTCTAAATACGAATTCATAAAAATTGAAA |
| | | 3516E3-F6 | GAATAAGTTGTTATGAGTTAATTTTCAAGA |
| | | 3516E3-F7 | TAAATAAAATTAACAAAAATGCAATCTAAAAGAA |
| | | 3516E3-F8 | CACAGCATCAAAATTGTTAGC |
| | | 3516E3-F9 | CCGTGCTGGTTCAAG |
| | | 3516E3-F10 | GAGGGGCTAGTGGG |
| | | 3516E3-F11 | GGAAGTGGTAGACACGC |
| | | 3516E3-F12 | AAAAAATATAATGGTTAATAGTGCTGTG |
| | | 3516E3-F13 | GTAATTAAAAAAAATAAAAAAGTTGACAAAAATT |
| | | 3516E3-F14 | CGCTGTAATAGCAACAACAATAATA |
| | | 3516E3-F15 | AATAATATTTTCAAAAATAAAAATAATTATATTTGCAA |
| | | 3516E3-F16 | CTCTAAGCTTCAAACTAGGTCA |
| | | 3516E3-F17 | AACTTTGCTCTCAATAGTTGTTT |
| | | 3516E3-F18 | TATGAAATCTAATCTATTTATTGTTTCTGACT |
| | | 3516E3-F19 | GCAATATTTATGTCAGCAGGAA |
| | | 3516E3-F20 | GAAGGTTTATACCCTTTGGAG |
| | | 3516E3-F21 | TGGTCAATATGGGGTATTAACTTTAT |
| | | 3516E3-F22 | CAATAACCTGCTTGACAAAATAAATTA |
| | | 3516E3-F23 | GGAAATTAATGGGAAATAAATTATTTAAAAACA |
| | | 3516E3-F24 | AGACATAATATCTTTTTACATTGGGAAA |
| | | 3516E3-F25 | TTTAGGAATTTTTTGGGGAGC |
| | | 3516E3-R1 | TGAGGGTGAGTTCCTGT |
| | | 3516E3-R2 | TTGTTTTTTAAACTTATTAATATTTTCTTCTGTAC |
| | | 3516E3-R3 | GGCAAACCCCAAAGC |
| | | 3516E3-R4 | GTGTTCTAATTTCTCGATCCC |
| | | 3516E3-R5 | ACTGTGTCCATTTATAGTAATTCTC |
| | | 3516E3-R6 | CTAGCCCTTTTTTATATAATTGCAGG |
| | | 3516E3-R7 | GTACCATACAGGCATTTCTTTTA |
| | | 3516E3-R8 | CTAGTACCGTTCCAAGCT |
| | | 3516E3-R9 | GTCCATCTGAAGTTTGAATAATCTC |
| | | 3516E3-R10 | ACGCTGTAAGATCCTCTC |
| | | 3516E3-R11 | GTAATTTTTAAAACCCACTGTCTTAAATA |
| | | 3516E3-R12 | CGTCTAGCAATCTTTCAGC |
| | | 3516E3-R13 | AGATTCAGGCCATTCTAATTCT |
| | | 3516E3-R14 | TCCAATTTCGCTGCATTTC |
| | | 3516E3-R15 | AATTCAATTTCAACTCCTGTTGAT |
| | | 3516E3-R16 | TTTTATCGCTGTGGCCTT |
| | | 3516E3-R17 | CTTTACAACAAGGCCAGAC |
| | | 3516E3-R18 | CGATCACTAAATATGTGATGCC |
| | | 3516E3-R19 | GTTGTTCTTTGTTATTTTTTCTATTAGTTTATT |
| | | 3516E3-R20 | CTTCGTGCTTTACATATTTTAAAACATT |
| | | 3516E3-R21 | GAGAAGTCCTATTAAGATCGCT |
| | | 3516E3-R22 | CTGTGAAAACTCCCGATTTATC |
| | | 3516E3-R23 | CATTTGTTATTGGATGAAATGCG |
| | | 3516E3-R24 | GCTTCCAACCCAAATTG |
| | | 3516E3-R25 | CGGTTCCGTAAGTTCCT |
| BCT3517 | flaB | 3517E3-F1 | TCTGCTTCTCAAAATGTAAGAACA |
| | | 3517E3-F2 | TAACCAAATGCACATGTTATCAAA |
| | | 3517E3-F3 | TTGCTGATCAAGCTCAATAT |
| | | 3517E3-F4 | GCAACTTACAGACGAAATTAAT |
| | | 3517E3-F5 | AGACAGAGGTTCTATACAAATTGA |
| | | 3517E3-F6 | AGGTAACGGCACATATTCAGA |
| | | 3517E3-F7 | TAAGAATGAAGGAATTGGCAGTT |
| | | 3517E3-F8 | AATTTAAATGAAGTAGAAAAAGTCTTAGT |
| | | 3517E3-F9 | GGCTATTAATTTTATTCAGACAACAGA |
| | | 3517E3-F10 | TTGTCACAAGCTTCTAGAAATA |
| | | 3517E3-F11 | TTTCTGGTAAGATTAATGCTCAAAT |
| | | 3517E3-F12 | GAGCTTCTGATGATGCTGCT |
| | | 3517E3-F13 | GAAAAGCTTTCTAGTGGGTAC |
| | | 3517E3-F14 | CATTAACGCTGCTAATCTTAGTAA |
| | | 3517E3-F15 | CATCAGCTATTAATGCTTCAAGA |
| | | 3517E3-F16 | CATGGAGGAATGATATATGATTATCATG |
| | | 3517E3-F17 | TTTTTTTTTAATTTTTGTGCTATTCTTTTTAAC |
| | | 3517E3-F18 | TAATAATAATTATTTTTAATGCTATTGCTATTTGC |
| | | 3517E3-F19 | ATTAAAGGCTTTTGATTTTAATCAAAGA |
| | | 3517E3-F20 | TTAAGCGCATGAAAGATCAAG |
| | | 3517E3-F21 | GTGGAAGGTGAACTTAATACC |
| | | 3517E3-F22 | GATTATAAAAAGAAGTACGAAGATAGAGAG |
| | | 3517E3-F23 | TTATTTTTTTGATTAAAAATTTTCAAGTCGTAA |
| | | 3517E3-F24 | GCTTCCGGAGGAGTTATTTAT |
| | | 3517E3-F25 | TAGGAGATTGTCTGTCGC |
| | | 3517E3-R1 | GCAACATTAGCTGCATAAATAT |
| | | 3517E3-R2 | TCCCTCACCAGAGAAAG |
| | | 3517E3-R3 | ACACCCTCTTGAACCGGTG |
| | | 3517E3-R4 | TGAGAAGGTGCTGTAGCAGG |
| | | 3517E3-R5 | TTGTAACATTAACAGGAGAATTAACTC |
| | | 3517E3-R6 | TTAGCAAGTGATGTATTAGCATCA |
| | | 3517E3-R7 | TGATCACTTATCATTCTAATAGCATTT |
| | | 3517E3-R8 | CTATTTTGGAAAGCACCTAAAT |
| | | 3517E3-R9 | GCATACTCAGTACTATTCTTTATAGAT |
| | | 3517E3-R10 | TGAGCATAAGATGCTTTTAGATTT |
| | | 3517E3-R11 | TCTGTCATTGTAGCATCTTTTA |
| | | 3517E3-R12 | TTAAAATACTATTAGTTGTTGCTGCTAC |
| | | 3517E3-R13 | ATTAGCCTGCGCAATCAT |
| | | 3517E3-R14 | GCAATGACAAAACATATTGGG |
| | | 3517E3-R15 | TTAATACAATTTATACCAATTAAACTAGAATTTT |
| | | 3517E3-R16 | ATAAAAAAACAAAAGATCCTTTAAAGGATC |
| | | 3517E3-R17 | ATAAATTATACTAAAATTATTAAATTTTTGCCGAT |
| | | 3517E3-R18 | GCCTGCATTATGCTTTATAACA |
| | | 3517E3-R19 | CCTACTCAAAGCAAACTCC |
| | | 3517E3-R20 | CGAAAATACTTTATAACAATCTTTAATTTTAACA |
| | | 3517E3-R21 | TCGACTTATCTGCTTTTTGTTAAC |
| | | 3517E3-R22 | CTATCTTTGCCATCTTCATAGTC |
| | | 3517E3-R23 | GCAATAAAAATAGAAGATTCTTTGTAGAT |
| | | 3517E3-R24 | TAAAATTTCATTTTCATAAACATCAAGATTAATA |
| | | 3517E3-R25 | GCCCGACATACCCA |
| BCT3518 | ospC | 3518E3-F1 | TTAATGAAAAAGAATACATTAAGTGCAATAT |
| | | 3518E3-F2 | CTAATAATTCATAAATAAAAAGGAGGCAC |
| | | 3518E3-F3 | TTTTCAAATAAAAAATTGAAAAACAAAATTGT |
| | | 3518E3-F4 | AATATTTATTCAAGATATTGAAGAATTTGAAAAA |
| | | 3518E3-F5 | TTTAAAATCAAATTAAGACAATATTTTTCAAATTC |
| | | 3518E3-F6 | AGCATATTTGGCTTTGCTTATG |
| | | 3518E3-F7 | AAATTAAAACTTTTTTTATTAAAGTATACTTCATTTAA |
| | | 3518E3-F8 | GCCTGAGTATTCATTATATAAGTCC |
| | | 3518E3-F9 | TATATTGGGATCCAAAATCTAATACAAG |
| | | 3518E3-F10 | CAATTTCTCTAATTCTTCTTGCAATTAG |
| | | 3518E3-F11 | GGAGTATAGTAAGGTATTACTTTTGTATAAA |
| | | 3518E3-F12 | TTCCTGAGATATTCATATTTTTAATTTCTTTT |
| | | 3518E3-F13 | GCAGGACTTCCACTTAGTA |
| | | 3518E3-F14 | GGTAGGAGCTTCTTTTGAATAAAC |
| | | 3518E3-F15 | CAAAATAGGTATTTTCAAATTAAAAATTTCCATA |
| | | 3518E3-F16 | AATTTAACAATTATTTGCATTCCATAACATA |
| | | 3518E3-F17 | GCTTAGAGTCTTTAGATACTAGGC |
| | | 3518E3-F18 | AAAGATTTCAGAGCTCCCATAT |
| | | 3518E3-F19 | TTCTGAAAATAAAAGAGATTTTTCATCTC |
| | | 3518E3-F20 | TGACTCATGATAATTTGAAATTTGTTTG |
| | | 3518E3-F21 | AAATTATCAGGCCTTTTTTCAATACTGTC |
| | | 3518E3-F22 | GCAATACAATTTTTTGTAAAAGCTAATTG |
| | | 3518E3-F23 | CCGTAAATTTTTTGAGTTTCATTTGAT |
| | | 3518E3-F24 | AGTTACTTCTGGATGGAATTGT |
| | | 3518E3-F25 | AATTTTTAATTATTTGATCACCAAATTCAG |
| | | 3518E3-R1 | ACCGCATTAGAATCCGTAAT |
| | | 3518E3-R2 | ACCTCTTTCACAGCAAGTT |
| | | 3518E3-R3 | CATCTATAGATGACAGCAACG |
| | | 3518E3-R4 | TTACCAATAGCTTTAGCAGCA |
| | | 3518E3-R5 | GTATCCAAACCATTATTTTGGTGTA |
| | | 3518E3-R6 | GCTAACAATGATCCATTGTGATTAT |
| | | 3518E3-R7 | TATTAGGGTTGATATTGCATAAGC |
| | | 3518E3-R8 | CTTCATTTTTCAATCCATCTAATTTTTG |
| | | 3518E3-R9 | TTAGCCGCATCAATTTTTTCC |
| | | 3518E3-R10 | TCTTTTAATTTATTAGTAAATGTTTCAGAACA |
| | | 3518E3-R11 | CTTCTTTACCAAGATCTGTGTG |
| | | 3518E3-R12 | CTTTTGCATCAGCATCAGT |
| | | 3518E3-R13 | TTTAGTTTTAGTACCATTTGTTTTTAAAATG |
| | | 3518E3-R14 | GATTCAAATAATTTTCCAAGTTCTTCAG |
| | | 3518E3-R15 | CTGCTTTTGACAAGACCTC |
| | | 3518E3-R16 | TTTAACTGAATTAGCAAGCATCTC |
| | | 3518E3-R17 | CCACAACAGGGCTTG |
| | | 3518E3-R18 | GATCTTAATTAAGGTTTTTTTGGACTT |
| | | 3518E3-R19 | CCAGTTACTTTTTTAAAACAAATTAATCTTATA |
| | | 3518E3-R20 | AGAAATCTTTCTTGACTTATATTGACTTT |
| | | 3518E3-R21 | GAATTTTAAGAAATTTTTTGAGAAAATAAAAAAATAAAA |
| | | 3518E3-R22 | TATTCTTTAAGAGAAGAGCTTAAAGTT |
| | | 3518E3-R23 | AAATTCAATTTATTAACGGCTTTTGTAATA |
| | | 3518E3-R24 | TCTAGCACCCAATTTTGTTTATATTTA |
| | | 3518E3-R25 | GTTTAAGCCTACTTAAAGTCTTTAAAATC |
| BCT3519 | hbb | 3519-20E3-F1 | CCCACACTCTCTCTTTCAAA |
| and | | 3519-20E3-F2 | GATATTAACCGGCATTTAACCTT |
| BCT3520 | | 3519-20E3-F3 | TCTAGCTTACAATCCCATTTATAAGA |
| | | 3519-20E3-F4 | CCTTCAAATTTTAATTTTCCTCTAAAAGTTA |
| | | 3519-20E3-F5 | CCTTCAAAAGAAGAATCAAGATACAA |
| | | 3519-20E3-F6 | CACACCCCCTTTTGAAGATA |
| | | 3519-20E3-F7 | GTAATAACCTTACTATTCTTGCCAATA |
| | | 3519-20E3-F8 | TTCTACTATTAATGTATCACAAATTACCAC |
| | | 3519-20E3-F9 | GCATTTACATTGCCCTTCAA |
| | | 3519-20E3-F10 | CAACCGCTGTTTAAATAAACCTT |
| | | 3519-20E3-F11 | AATATTTTTTTTGTTTTTACATCCCCATAT |
| | | 3519-20E3-F12 | CACACTTACCATCAAAAATTATATTATCAT |
| | | 3519-20E3-F13 | AAGAAAATAAATCTACAATTTCATTAGACTTTA |
| | | 3519-20E3-F14 | CAAAGTATCTTTTATTTGTGAAACGG |
| | | 3519-20E3-F15 | TCTACTTATTATTAATTAATAAAAAACACTGACC |
| | | 3519-20E3-F16 | CTCTACGAATTAAATTTTTAAGAAAGGATTTTA |
| | | 3519-20E3-F17 | ATCAAATCCACCATTTTTTTTATCCA |
| | | 3519-20E3-F18 | CCAACCGCCTTATTTCAC |
| | | 3519-20E3-F19 | TTTTCAAATTATCTTCAATCTTAAACTCTTTAG |
| | | 3519-20E3-F20 | TTTTAGCAACAACTTTAACCACTTT |
| | | 3519-20E3-F21 | TGTCACGCTAGATGCAG |
| | | 3519-20E3-F22 | CTTTACGCCACTTAAATCTGC |
| | | 3519-20E3-F23 | AATCAGAAAATATTACCCCGTTTG |
| | | 3519-20E3-F24 | ATATTATTTTCTAAACCTGAAGAAGGAATAT |
| | | 3519-20E3-F25 | CATTAAAAAATTTGATGATATTACTTTGCTC |
| | | 3519-20E3-R1 | GTTTTGCTGTTAAAGTAAGGAAATTAG |
| | | 3519-20E3-R2 | GCTGCTAGAAAAAAATCTCGTT |
| | | 3519-20E3-R3 | CTGCTAGAAAGCGAATAATTCATAA |
| | | 3519-20E3-R4 | GAATTTTTTAAATTTGTTGCAAAAAAACTAG |
| | | 3519-20E3-R5 | GCGGGTAAGAAAGACGAA |
| | | 3519-20E3-R6 | GAAAAACGCTGTATCAACATGA |
| | | 3519-20E3-R7 | ATTAGAAATGTAAGTGTAAAAAGTGAATTAAAA |
| | | 3519-20E3-R8 | CGCTCTCGTCAAAATTTAAAAAG |
| | | 3519-20E3-R9 | CTTGAGAAAAAATGCATCTGC |
| | | 3519-20E3-R10 | GATATATTAAAGCTATTGTTTAATAATATTATTAAGGA |
| | | 3519-20E3-R11 | ATTAACTTAAATCTTTGATTGACTATATTTGAAT |
| | | 3519-20E3-R12 | AGGTTTTTGAATATATTAATCAAAACTATTGT |
| | | 3519-20E3-R13 | ATTTTGAATAAAAAAATTTCTTATTCCATGC |
| | | 3519-20E3-R14 | CAAAGAAAATCATCAGACAAAAAAGG |
| | | 3519-20E3-R15 | GAATTTGAATTTAACAATAAAAATTATTTATGCTT |
| | | 3519-20E3-R16 | GAATTTTTTGAAAAAATTTTTATTGCCAG |
| | | 3519-20E3-R17 | CTGTGAAAGAAAAATTTTTAAAAGTGAAAT |
| | | 3519-20E3-R18 | CAATATAGTGTTATTTTATGAGTTTAGGAAAG |
| | | 3519-20E3-R19 | TTTTGTTGGGGGATTTTTCAG |
| | | 3519-20E3-R20 | TTTTGTTGGGGGATTTTTCAG |
| | | 3519-20E3-R21 | GCAATATATTTATTTTTTTATTTATTTGTTTTATTGATATTA |
| | | 3519-20E3-R22 | GTATTATGATTGCTTTATTTGTTTATTACATTTC |
| | | 3519-20E3-R23 | GATATTATTTATCTTGTACTTATCTTTTTATGTTTT |
| | | 3519-20E3-R24 | GTCCCAAAATTGGAAAATTTTCC |
| | | 3519-20E3-R25 | TATTTAAAGAGCTTAAAATTAAGAGAAAAGATC |
| BCT3511 | gyrB | 3511E3-F1 | CGTGAAGCTGCAAGAAAA |
| | | 3511E3-F2 | TGGAAAAGCAATAAAAGCTGCTG |
| | | 3511E3-F3 | TGTTGTATATGAACATTTATTGGAAAT |
| | | 3511E3-F4 | GCTTGGTAATTCTGAGATAAGAAA |
| | | 3511E3-F5 | CCTCAATTTGAAGGTCAAACAAA |
| | | 3511E3-F6 | ATTTTAAAGAGGGGCTTACAGCT |
| | | 3511E3-F7 | GCCATGAATGAAGCTTTTAAA |
| | | 3511E3-F8 | CTCATGTTATGGGATTTAGAAGTGG |
| | | 3511E3-F9 | CTGACAACATTCTTCTTTTGTTAA |
| | | 3511E3-F10 | TGTTAATGTGGGGCTTAAATG |
| | | 3511E3-F11 | GCTTTTCAATCAGAACCTTATT |
| | | 3511E3-F12 | GAGGGTGGGATAAAATCTTTTT |
| | | 3511E3-F13 | TGGTAAAGAAAAATCTTCAAAATTTTAT |
| | | 3511E3-F14 | CGATAAAATATACATTTCAATTGAAGATAA |
| | | 3511E3-F15 | GGCTTAAAGAGCTTGCTTTT |
| | | 3511E3-F16 | AGATTATAATTTCGATGTTCTTGAAAAA |
| | | 3511E3-F17 | CGGATTCTGAAATTTTTGAAACTTT |
| | | 3511E3-F18 | GGGGACTAAGGTTACTTTTTT |
| | | 3511E3-F19 | AGAAGTTGTGGGGGAATCTTCTGTT |
| | | 3511E3-F20 | CTTTTTCAAAAGGTATTCCGACTT |
| | | 3511E3-F21 | GGTTTATGTTAATAGAGATGGAAAAAT |
| | | 3511E3-F22 | GGTTGTAAATGCTCTATCTTCGTT |
| | | 3511E3-F23 | TGGTAAGTTTAATAAAGGCACGTAT |
| | | 3511E3-F24 | CCTTGAACTTGTTTTAACAAAATTAC |
| | | 3511E3-F25 | ACCGATATTCATGAAGAGGAG |
| | | 3511E3-R1 | TACCCATTTTAGCACTTCCTCCA |
| | | 3511E3-R2 | TGGCAAAATGGCCTGAAAAA |
| | | 3511E3-R3 | TTGTTTTCTCAACATTAAGCATTTT |
| | | 3511E3-R4 | ATCATTGGTGATAACCTTATCTTCT |
| | | 3511E3-R5 | ACTCCTGCACCAAGAGAT |
| | | 3511E3-R6 | ATCTTGTGATAACGAAGTTTTGTA |
| | | 3511E3-R7 | TCCATCAACATCGGCATCTG |
| | | 3511E3-R8 | AAAAGCTAAAAGCAAAGTTCTAAT |
| | | 3511E3-R9 | ATATATCCATTTTCAATTAAATCTCTCAT |
| | | 3511E3-R10 | TATAAAGAGGAGGCATGGCT |
| | | 3511E3-R11 | TAAAAATAATAAATACGATTGTCATACTTT |
| | | 3511E3-R12 | TATTGCGATTTTTAGTTTCAATAGAA |
| | | 3511E3-R13 | CCCAAGCCCTTTATATCTCTGAA |
| | | 3511E3-R14 | AGCTGCGTTGGATTCATC |
| | | 3511E3-R15 | CTAGCAGGATCCATAGTTGTTT |
| | | 3511E3-R16 | ATCATCTATATTCATCAATCTCATTTTT |
| | | 3511E3-R17 | GAGTAACAAAAATTTTTTCAGCTTCA |
| | | 3511E3-R18 | TTTCTGGGCTCAACTAAATCT |
| | | 3511E3-R19 | GATTAATTACATTAAGTGCATTCTGTTC |
| | | 3511E3-R20 | CCATTAACGCTCCAATTACAC |
| | | 3511E3-R21 | TCCTAACATTTAATATTTGTTCTTTATTTTC |
| | | 3511E3-R22 | GCATAATTTAAATAAGAAGTTTTTATTTCATCT |
| | | 3511E3-R23 | GAAGAGCTCTAGAAACAATAACTGA |
| | | 3511E3-R24 | TGGTTTAAGACCATCTCTTACGT |
| | | 3511E3-R25 | CTCATACATAGAATAAAGTATTCTCCTG |

Since two of the target regions are close a single set of flanking oligos was designed to cover both targets. All primers were brought up to an initial concentration of 1 mM in 10 mM Tris pH 8.0 and 50 uM EDTA (pH 8.0). The primers were mixed in equal proportions to create a 1 mM oligo mix. Primers were designed using B. burgdorferi B31 genome sequence (gi|15594346) and have a GC content of 25 to 50%, spaced 6-10 nucleotides apart and have a target TM of 52° C. To ensure removal of any contaminating salts the pooled primers were dialyzed twice for 4 hours at 4° C. against a 4 L solution containing 10 mM Tris pH 8.0 and 50 uM EDTA pH 8.0 using a 5 mL Float-A-Lyzer G2 (Spectrum Laboratories, Rancho Dominguez, Calif.) with a molecular weight cutoff of 0.5-1 kDa.

The nested isothermal amplification was performed in a 225 ul reaction in a 0.6 mL PCR tube (Axygen Inc., Union City, Calif.) containing 200 uL of nucleic acid extract, 22.5 ul Ibis 10× PCR Buffer II²¹ (Ibis Biosciences, Carlsbad, Calif.), 0.2 uM dNTPs (Bioline, Tauton, Mass.), and 10 uM oligo mix. The reactions were incubated at 95° C. for 10 min followed by a cooling to 56° C. in a MJ Thermocycler (Bio-Rad Laboratories, Hercules, Calif.). The reactions were then removed to a heat block at 56° C. and 11.25 U of BstE DNA polymerase (Lucigen, Middleton, Wis.) enzyme added and the reactions incubated at 56° C. for 2 hours followed by an 80° C. heat inactivation for 20 min. The resulting reaction was used directly in the subsequent PCR without purification.

Detection of B. burgdorferi from Whole Blood from Patients with Clinically Diagnosed Early Lyme Disease.

Borrelia was detected by processing 2 ul of Borrelia enriched nucleic acid extracts per PCR reaction on a previously described broad-range PCR/ESI-MS assay designed to detect and characterize Borrelia burgdorferi as previously described.²¹. Electrospray ionization mass spectrometry was performed on the PLEX-ID biosensor (Abbott Molecular). Briefly, after PCR amplification, 30 μL aliquots of each PCR reaction were desalted and analyzed by mass spectrometry as previously described²¹⁻²³ (herein incorporated by reference as if fully set forth herein). Analysis of amplicons from any one of the eight primer pairs in the assay can be used to positively identify Borrelia DNA in a specimen.

### Results

The data are supportive that this new strategy can positively impact current clinical and laboratory impediments to early diagnosis of Lyme disease. A serendipitous finding of clinical importance was that the majority (˜60%), of rashes were not the classic EM in PCR positive cases.

Detection of Lyme disease in 29 patients with classic and non-classic EM Examination of nucleic acid extract from 1.25 ml of whole blood from the independent set of 29 endemic patients with clinically diagnosed Lyme disease with classic or non-classic EM and 44 healthy controls showed that 14 of the 29 Lyme patients, and 0 of 45 controls, (48%, p=0.0001) had detectable B. burgdorferi. The pre-PCR enrichment step enabled increased detection of Borrelia from 2 to 14 cases (7×) when assessed by the PCR/ESI-MS assay. Two-tiered serology was positive in 14 of the 29 specimens, not all of which overlapped with the 14 specimens positive by NIA/PCR/ESI-MS from Table 2. Nine of the clinically defined early Lyme disease specimens tested negative by both PCR and serology assays. However, we did not have skin isolates to determine if the Borrelia was actually present as a skin-restricted strain which did not disseminate to blood.^{23,24} Table 2 is presented to show PCR detection is possible even when serology is negative or not yet positive. The data support the concept that unambiguous diagnosis can be made early, before seroconversion.

TABLE 2
EM, B. burgdorferi PCR and serological analysis of 14 Early Lyme
disease specimens positive by NIA/PCR/ESI-MS from whole blood.

| | | PCR | Early 2-tier | | | | Follow-up two-tier | | | |
|---|---|---|---|---|---|---|---|---|---|---|
| | EM | PCR | | | # IgM | # IgG | | | # IgM | # IgG |
| ID | EM¹ | Result | Result² | ELISA | bands | bands | Result² | ELISA | bands | bands |
| 26 | NC | Pos | Neg | ≦0.90 | — | — | Pos | 4.35 | 3 | 1 |
| 33 | C | Pos | Pos | >5.00 | 3 | 4 | ND | — | — | — |
| 37 | C | Pos | Neg | 1 | 1 | 3 | Pos | >5.00 | 3 | 5 |
| 40 | NC | Pos | Pos | >5.00 | 3 | 4 | ND | — | — | — |
| 44 | NC | Pos | Neg | ≦0.90 | — | — | Pos | >5.00 | 3 | 6 |
| 45 | NC | Pos | Neg | 1.36 | 1 | 1 | Pos | >5.00 | 2 | 2 |
| 46 | NC | Pos | Pos | >5.00 | 3 | 1 | ND | — | — | — |
| 47 | C | Pos | Pos | >5.00 | 3 | 4 | ND | — | — | — |
| 48 | NC | Pos | Neg | ≦0.90 | — | — | Neg | 1.46 | 1 | 2 |
| 49 | NC | Pos | Pos | 2.49 | 2 | 0 | ND | — | — | — |
| 51 | C | Pos | Neg | 4.49 | 1 | 3 | Neg | >5.00 | 2 | 4 |
| 52 | NC | Pos | Pos | 3.54 | 2 | 3 | ND | — | — | — |
| 53 | NC | Pos | Pos | 4.73 | 3 | 2 | ND | — | — | — |
| 54 | NC | Pos | Pos | 2.86 | 2 | 2 | ND | — | — | — |

¹C = classic erythema migrans; NC= non-classic erythema migrans
²Positivity based upon CDC two tiered serologic criteria
Results illustrated for 14/29 patients who were PCR+.
Pos = positive, Neg = negative, ND = not done

Of the 14 early Lyme specimens that tested positive by the NIA/PCR/ESI-MS assay, 6 were negative by the 2-tiered test at the time of presentation before the initiation of antibiotics (Table 2). Of these 6 specimens, 4 seroconverted by the follow-up serological testing 3 weeks later indicating that these were likely recent infections. Two of the NIA/PCR/ESI-MS positive specimens (#48 and #51) did not seroconvert sufficiently to meet the CDC surveillance criteria²⁵ but both specimens showed an increase in ELISA titers and increased IgG/IgM blot reactivity, indicating that these patients were most likely represent weak or slow responders possibly due to antibiotic therapy²⁶ or for unknown reasons. Of the 44 control patient sera, a single sample was seropositive by ELISA and IgG western blot, but was PCR negative. This low rate in our sample set from the eastern United States is consistent with the known background seroreactivity from remote exposure in an endemic region.

These results demonstrate that B. burgdorferi infection can be diagnosed early, and even prior to seroconversion, from whole blood in a high percentage of patients with suspected Lyme using a pre-PCR Borrelia-DNA enhancing method coupled with PCR/ESI-MS assay. Other isothermal amplification methods to generate genetic material has been described but not for Borrelia.^{7,8} In this case, a novel nested isothermal amplification (NIA) was coupled with a PCR/ESI-MS assay and results were contrasted with the two-tier serology. However, in a clinical situation, finding Borrelia DNA circulating in blood in an ill person with endemic area exposure will have such a strong possibility of a current infection that it would support a clinical decision to treat regardless of whether there is a classical EM, an atypical skin lesion, or extracutaneous systemic symptoms and signs suggestive of Lyme disease. The could allow for early diagnosis of the 20-30% of patients with early Lyme disease that present with “viral-like” symptoms indistinguishable for other community acquired infectious diseases. In contrast, a single serologic test, even if positive, may be difficult to interpret in a person living in an endemic area because this single result could represent past exposure. The present strategy has the potential to remove this ambiguity.

Work conducted during development of embodiments of the present invention led to the conclusion that hold blood may be preferable to serum or plasma for detection of Borrelia. While not limited to any mechanism, this may be due to the pathogen adhering to particular blood cells. In contrast to many diagnostics based on analysis of small (ul) of samples, the present disclosure is configured to handle larger volumes (mls), but within clinical practice volumes, to increase the detection of pathogens that may be present only in low copy numbers. A recent paper²⁷ showed that B. burgdorferi may be present in a high percentage of patients with Lyme disease but may require “teasing” it out with combinations of cultures and PCR. Although this method is not as easily performed within a clinically useful time period, as that which is described herein, it does suggest that there is an attainable target should it be present.

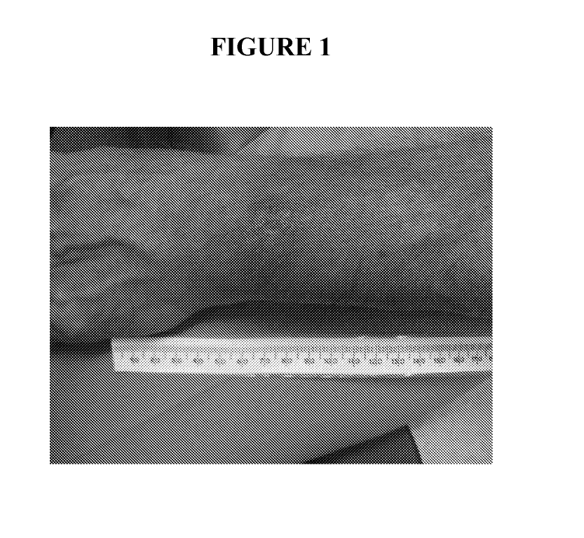

A serendipitous observation in this Example and an important clinical finding was the high percentage (57%) of PCR positive patients whose skin rash was not the classical EM but an atypical lesion (see FIG. 1) that could be easily misdiagnosed. Photographs of lesions (all greater than 5 cm) were presented to four physicians experienced with Lyme disease, including two dermatologists. Without knowledge of the laboratory data they were asked to categorize the lesions into 1) those that they would expect any reasonably trained physician to recognize as EM and 2) into those that they would not fault such a physician from referring the patient or desiring supportive laboratory tests or more time for observation.^{28,29} Based on previous reports, including a vaccine trial, of atypical rashes in 25-30% of microbiologically confirmed B. burgdorferi infected subjects^{4,5} it was expected a similar percentage, but were surprised by almost 60%. This high percentage serves as an alert for physicians to consider Lyme disease in the differential diagnosis of patients with endemic area exposure and a rash that is not the classic EM. These atypical lesions serve as a reminder of the need for diagnostics for the endemic area patient who only has a flu-like illness during the high season for Lyme disease. Such a patient may benefit from the testing strategy described in this Example or, at the very least, closer monitoring.

It is believed that these results demonstrate a useful strategy to the diagnosis of infections when there is a paucity of pathogens or the immune response cannot provide an unambiguous result. It is likely that many of these difficult to diagnose infections will require a battery of diagnostic platforms each with advantages depending on when the patient presents.

### REFERENCES

1 Benach J L, Bosler E M, Hanrahan J P et al. Spirochetes isolated from the blood of two patients with Lyme disease. N Engl J Med 1983; 308:740-2.
2 Steere A C. Lyme disease. N Engl J Med 1989; 321:586-96.
3 Steere A C. Lyme disease. N Engl J Med 2001; 345:115-25.
4 Steere A C, Sikand V K, Meurice F et al. Vaccination against Lyme disease with recombinant Borrelia burgdorferi outer-surface lipoprotein A with adjuvant. Lyme Disease Vaccine Study Group. N Engl J Med 1998; 339:209-15.
5 Berger B W, Johnson R C, Kodner C, Coleman L. Cultivation of Borrelia burgdorferi from erythema migrans lesions and perilesional skin. J Clin Microbiol 1992; 30:359-61.
6 Crowder C D, Rounds M A, Phillipson C A et al. Extraction of total nucleic acids from ticks for the detection of bacterial and viral pathogens. J Med Entomol 2010; 47:89-94.
7 Mori Y, Notomi T. Loop-mediated isothermal amplification (LAMP): a rapid, accurate, and cost-effective diagnostic method for infectious diseases. J Infect Chemother 2009; 15:62-9.
8 Notomi T, Okayama H, Masubuchi H et al. Loop-mediated isothermal amplification of DNA. Nucleic Acids Res 2000; 28:E63.
9 Crowder C D, Matthews H E, Schutzer S et al. Genotypic variation and mixtures of Lyme Borrelia in Ixodes ticks from North America and Europe. PLoS ONE 2010; 5:e10650.
10 Cox-Foster D L, Conlan S, Holmes E C et al. A metagenomic survey of microbes in honey bee colony collapse disorder. Science 2007; 318:283-7.
11 Briese T, Palacios G, Kokoris M et al. Diagnostic system for rapid and sensitive differential detection of pathogens. Emerg Infect Dis 2005; 11:310-3.
12 Eckburg P B, Bik E M, Bernstein C N et al. Diversity of the human intestinal microbial flora. Science 2005; 308:1635-8.
13 Ecker D J, Sampath R, Blyn L B et al. Rapid identification and strain-typing of respiratory pathogens for epidemic surveillance. Proc Natl Acad Sci USA 2005; 102:8012-7.
14 Hofstadler S A, Sampath R, Blyn L B et al. TIGER: the universal biosensor. Intl J Mass Spectrometry 2005; 242:23-41.
15 Jacobs M R, Bajaksouzian S, Bonomo R A et al. Occurrence, distribution and origins of serotype 6C Streptococcus pneumoniae, a recently recognized serotype. J Clin Microbiol 2008.
16 Ecker D J, Sampath R, Massire C et al. Ibis T5000: a universal biosensor approach for microbiology. Nat Rev Microbiol 2008; 6:553-8.
17 Hannis J C, Manalili S M, Hall T A et al. High-resolution genotyping of Campylobacter species by use of PCR and high-throughput mass spectrometry. J Clin Microbiol 2008; 46:1220-5.
18 Blyn L B, Hall T A, Libby B et al. Rapid detection and molecular serotyping of adenovirus by use of PCR followed by electrospray ionization mass spectrometry. J Clin Microbiol 2008; 46:644-51.
19 Eshoo M W, Whitehouse C A, Zoll S T et al. Direct broad-range detection of alphaviruses in mosquito extracts. Virology 2007; 368:286-95.
20 Sampath R, Russell K L, Massire C et al. Global surveillance of emerging Influenza virus genotypes by mass spectrometry. PLoS ONE 2007; 2:e489.
21 Sampath R, Hall T A, Massire C et al. Rapid identification of emerging infectious agents using PCR and electrospray ionization mass spectrometry. Ann N Y Acad Sci 2007; 1102:109-20.
22 Sampath R, Hofstadler S A, Blyn L B et al. Rapid identification of emerging pathogens: coronavirus. Emerg Infect Dis 2005; 11:373-9.
23 Seinost G, Dykhuizen D E, Dattwyler R J et al. Four clones of Borrelia burgdorferi sensu stricto cause invasive infection in humans. Infect Immun 1999; 67:3518-24.
24 Wormser G P, Liveris D, Nowakowski J et al. Association of specific subtypes of Borrelia burgdorferi with hematogenous dissemination in early Lyme disease. J Infect Dis 1999; 180:720-5.
25 CDC. Recommendations for test performance and interpretation from the Second National Conference on Serologic Diagnosis of Lyme Disease. MMWR Morb Mortal Wkly Rep 1995; 44:590-1.
26 Dattwyler R J, Volkman D J, Luft B J, Halperin J J, Thomas J, Golightly M G. Seronegative Lyme disease. Dissociation of specific T- and B-lymphocyte responses to Borrelia burgdorferi. N Engl J Med 1988; 319:1441-6.
27 Liveris D, Schwartz I, Bittker S et al. Improving the Yield of Blood Cultures in Early Lyme Disease. Journal of Clinical Microbiology 2011; JCM.
28 Berger B W. Erythema migrans. Clin Dermatol 1993; 11:359-62.
29 Berger B W. Dermatologic manifestations of Lyme disease. Rev Infect Dis 1989; 11 Suppl 6:S1475-S1481.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method of nucleic acid extraction comprising:
a) contacting a sample whole blood with beads, a proteinase, and an anionic surfactant to generate a treated sample;
b) homogenizing said treated sample to generate a cell lysate;
c) centrifuging said cell lysate comprising a supernatant;
d) separating said supernatant from said cell lysate;
e) adding magnetic particles and lysis buffer to said supernatant to generate a magnetic-particle sample, wherein said magnetic particles are configured to bind nucleic acid molecules;
f) washing said magnetic-particle sample with a wash buffer;
g) treating said magnetic-particle sample in order to generate a dried magnetic bead sample; and
h) treating said dried magnetic bead sample with an elution buffer such that a purified nucleic acid sample is generated that comprises purified nucleic acid.

2. The method of claim 1, further comprising subjecting said purified nucleic acid to PCR and/or isothermal nested PCR to generate amplified nucleic acid.

3. The method of claim 2, further subjecting said amplified nucleic acid to mass spectrometry bioagent analysis in order to identify the source of said purified nucleic acid.

4. The method of claim 3, wherein said mass spectrometry bioagent analysis comprises electrospray ionization mass spectrometry and base composition analysis.

5. A method comprising:
a) contacting a sample comprising isolated nucleic acid with a buffer, dNTPs, and a plurality of nested PCR primer pairs configured to amplify at least part of at least one bioagent target sequence;
b) incubating said sample with a DNA polymerase under isothermal conditions such that amplified nucleic acid is generated;
c) inactivating said DNA polymerase; and
d) subjecting said amplified nucleic acid to mass spectrometry bioagent analysis in order to identify the source of said isolated nucleic acid.

6. The method of claim 5, wherein said mass spectrometry bioagent analysis comprises electrospray ionization mass spectrometry and base composition analysis.

7. The method of claim 5, wherein said DNA polymerase comprises BstE DNA polymerase.

8. The method of claim 5, wherein said incubating is conducted at about 56 degrees Celsius.

9. The method of claim 5, wherein said inactivating said DNA polymerase comprises heating said sample to at least about 80 degrees Celsius.

10. The method of claim 5, wherein said plurality of nested PCR primer pairs comprises at least 10 primer pairs.

11. The method of claim 5, wherein said plurality of nested PCR primer pairs comprises at least 20 primer pairs.

12. The method of claim 5, wherein said at least one bioagent target sequence comprises at least five bioagent target sequences.

13. The method of claim 5, further comprising a step after c) but before d) of further amplifying said amplified nucleic acid without any purification of said amplified nucleic acid.
